(19) United States
(12) Patent Application Publication
(10) Pub. No.: US 2026/0328773 A1
(43) Pub. Date: Oct. 22, 2026

(54) COMPOSITION AND METHOD FOR ISOLATING CONJUGATABLE BIOMOLECULES FROM BIOLOGICAL MEDIA

(71) Applicant: Life Technologies Corporation, Carlsbad, CA (US)

(72) Inventors: Ramesh Ganapathy, Rockford, IL (US); Marie Nlend, Rockford, IL (US); Douglas Anderson, Eugene, OR (US)

(73) Assignee: Life Technologies Corporation, Carlsbad, CA (US)

(21) Appl. No.: 19/641,370

(22) Filed: Apr. 7, 2026

Related U.S. Application Data
(60) Provisional application No. 63/786,189, filed on Apr. 9, 2025.

(51) Int. Cl. G01N 33/58; G01N 21/64; G01N 33/68

(52) U.S. Cl. CPC G01N 33/582; G01N 21/6428; G01N 33/6854; G01N 2021/6439

(57) ABSTRACT

Disclosed herein are aspects of a composition comprising an amine buffer and a matrix comprising (i) a porous size exclusion support and (ii) a cationic moiety for separating one or more small molecules from one or more large molecules in a sample using differences in one or more properties such as the size of the molecules, charge of the molecules, the isoelectric point (pI) of the molecules, and/or any combination of these properties including methods and kit aspects. Also disclosed herein are aspects of a method for isolating conjugatable antibodies from a sample, wherein the method does not require additional sample preparation steps for subsequent dye conjugation.

### CROSS REFERENCE TO RELATED APPLICATION

This application claims the benefit of and priority to the earlier filing date of U.S. Provisional Patent Application No. 63/786,189 filed on Apr. 9, 2025, the entirety of which is incorporated herein by reference.

### FIELD

The present disclosure concerns a method, composition, and kit for sample preparation, such as isolating conjugatable large molecules from small molecules.

### BACKGROUND

Sample preparation techniques for isolating biomolecules may require separating biomolecules from other sample components and from sample processing components to enable downstream analysis and processing of the biomolecule. During sample preparation of biomolecules, such as proteins or nucleic acids, there often is a need to label the biomolecules with dyes, affinity tags, radioactive labels, mass tags, and the like. In other instances, the biomolecules may need to be chemically modified, such as by reduction, oxidization, cross-linking, and/or alkylation.

After isolating biomolecules from other sample components, however, conventional methods include either a buffer exchange step using a desalting resin with a buffer prior to the addition of a dye. Alternatively, the isolated biomolecule can be spiked with one or more buffers prior to the addition of the dye. As such, conventional methods require an additional step in preparing the biomolecule before downstream dye conjugation, which leads to sample loss and/or dilution of the antibody.

Accordingly, there is a need for an improved purification method, composition, and kit for purifying proteins from samples that prevent sample loss and/or dilution of the antibody and avoid extraneous cleaning/buffering steps, thereby reducing sample preparation time and materials needed.

### SUMMARY

Disclosed herein is as method, comprising: combining an amine buffer according to Formula I with a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof, wherein Formula I is

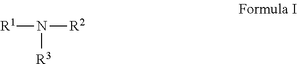

wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof.

Also disclosed herein is a method, comprising: exposing a sample to a matrix and a buffer, wherein (a) the matrix comprises (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof; and (b) the buffer is the amine buffer disclosed herein.

A kit is also disclosed herein, the kit comprising a first container comprising a matrix comprising (i) a porous size exclusion support, and (ii) one or more cationic moieties associated with the porous size exclusion support, wherein the matrix can separate one or more molecules in a sample by using a molecular weight of the one or more molecules, a charge of the one or more molecules, an isoelectric point (pI) of the one or more molecules, or a combination thereof; and a second container comprising the amine buffer disclosed herein.

A composition is also disclosed herein, the composition comprising the amine buffer disclosed herein; and a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof.

Also disclosed herein is a composition, comprising: a matrix (i) a porous size exclusion support, and (ii) one or more cationic moieties associated with the porous size exclusion support, wherein the matrix can separate one or more molecules in a sample by using a molecular weight of the one or more molecules, a charge of the one or more molecules, an isoelectric point (pI) of the one or more molecules, or a combination thereof; an antibody; and a means for equilibrating the antibody for conjugation, wherein the means does not comprise 2-amino-2-(hydroxymethyl) propane-1,3-diol, triethylammonium bicarbonate, sodium acetate, or 4-(2-hydroxyethyl)-1-piperazineethanesulfonic acid.

The foregoing and other objects, features, and advantages of the present disclosure will become more apparent from the following detailed description, which proceeds with reference to the accompanying figures.

### BRIEF DESCRIPTION OF THE DRAWINGS

FIG. 1A is a digital image of a fluorescent gel showing the recovery of Goat Anti Rabbit-DyLight® 650 (GAR-Dy 650) conjugate using non-spiked 25 mM bicine (Lane 1); 25 mM bicine spiked with 1 M borate (Lane 2); non-spiked 50 mM bicine (Lane 3); 50 mM bicine spiked with 1 M borate (Lane 4); non-spiked 75 mM bicine (Lane 5); 75 mM bicine spiked with 1 M borate (Lane 6); non-spiked 75 mM spodium acetate (Lane 7); and 75 mM sodium acetate spiked with 1 M borate (Lane 8); the results demonstrate that non-spiked and spiked bicine used at various concentrations produced conjugatable Goat Anti Rabbit antibody (GAR) and that subsequent conjugation had a desirable recovery of a GAR-Dy 650 conjugate.

FIG. 1B is a bar graph showing the GAR-Dy 650 recovery (%) of the fluorescent gel of FIG. 1A and demonstrating a 78% recovery for non-spiked 25 mM bicine (Lane 1); 86% recovery for 25 mM bicine spiked with 1 M borate (Lane 2); 99% recovery for non-spiked 50 mM bicine (Lane 3); 97% recovery for 50 mM bicine spiked with 1 M borate (Lane 4); 88% recovery for non-spiked 75 mM bicine (Lane 5); 99% recovery for 75 mM bicine spiked with 1 M borate (Lane 6); 51% recovery for non-spiked 75 mM sodium acetate (Lane 7); and 100% recovery for 75 mM sodium acetate spiked with 1 M borate (Lane 8).

FIG. 1C is a digital image of a fluorescent gel showing the recovery GAR-Dy 650 conjugate using 125 mM bicine spiked with 1 M borate (Lane 1); non-spiked 125 mM bicine (Lane 2); 150 mM bicine spiked with 1 M borate (Lane 3); 150 mM bicine spiked with 1 M borate (Lane 4); non-spiked 75 mM sodium acetate (Lane 5); and 75 mM sodium acetate spiked with 1 M borate (Lane 6); the results demonstrate that the non-spiked and spiked bicine buffers at higher concentrations produced conjugatable GAR and that subsequent conjugation had a desirable recovery of a GAR-Dy 650 conjugate.

FIG. 1D is a bar graph showing the GAR-Dy 650 recovery (%) of the fluorescent gel of FIG. 1C and demonstrating a 89% recovery for 125 mM bicine spiked with 1 M borate (Lane 1); 88% recovery for non-spiked 125 mM bicine (Lane 2); 93% recovery for 150 mM bicine spiked with 1 M borate (Lane 3); 98% recovery for 150 mM bicine spiked with 1 M borate (Lane 4); 31% recovery for non-spiked 75 mM sodium acetate (Lane 5); and 91% recovery for 75 mM sodium acetate spiked with 1 M borate (Lane 6).

FIG. 2A is a digital image of a fluorescent gel showing the recovery of GAR-Dy 647 conjugate (fluorescent stain) using non-spiked 125 mM bicine (Lane 1); non-spiked 125 mM tricine (Lane 2); and non-spiked 75 mM sodium acetate (Lane 3); the results demonstrate that the lanes using non-spiked bicine and non-spiked tricine produced conjugatable GAR and that subsequent conjugation to NHS DyLight® 647 exhibited a higher intensity relative to the lane using sodium acetate.

FIG. 2B is a bar graph showing the GAR-647 conjugate quantitation (fluorescence densitometry) results for the gel of FIG. 2A and resulting background corrected volumes for non-spiked 125 mM bicine (Lane 1); non-spiked 125 mM tricine (Lane 2); and non-spiked 75 mM sodium acetate (Lane 3).

FIG. 2C is a digital image of a fluorescent gel showing the recovery of GAR-Dy 800 conjugate (fluorescent stain) using non-spiked 125 mM bicine (Lane 1); non-spiked 125 mM tricine (Lane 2); and non-spiked 75 mM sodium acetate (Lane 3); the results demonstrate that the lanes using bicine and tricine produced conjugatable GAR and that subsequent conjugation to NHS DyLight® 800 exhibited a higher intensity relative to the lane using sodium acetate.

FIG. 2D is a bar graph showing the GAR-800 conjugate quantitation (fluorescence densitometry) results for the gel of FIG. 2C and resulting background corrected volumes for non-spiked 125 mM bicine (Lane 1); non-spiked 125 mM tricine (Lane 2); and non-spiked 75 mM sodium acetate (Lane 3).

FIG. 2E is a digital image of a Coomassie protein-stained gel showing the recovery of GAR-Dy 647 conjugate (protein stain) using non-spiked 125 mM bicine (Lane 1); non-spiked 125 mM tricine (Lane 2); and non-spiked 75 mM sodium acetate (Lane 3); results demonstrate that the lanes using bicine and tricine produced conjugatable GAR, with subsequent conjugation to NHS DyLight® 647, had similar recoveries when stained with Coomasie protein stain and therefore had similar amounts of conjugate present.

FIG. 2F is a bar graph showing the GAR-Dy 647 conjugate quantitation (fluorescence densitometry) results for the gel of FIG. 2E and resulting background corrected volumes for non-spiked 125 mM bicine (Lane 1); non-spiked 125 mM tricine (Lane 2); and non-spiked 75 mM sodium acetate (Lane 3).

FIG. 2G is a digital image of a Coomassie protein-stained gel showing the recovery of GAR-Dy 800 conjugate (protein stain) using non-spiked 125 mM bicine (Lane 1); non-spiked 125 mM tricine (Lane 2); and non-spiked 75 mM sodium acetate (Lane 3); results demonstrate that the evaluations using bicine and tricine produced conjugatable GAR, with subsequent conjugation to NHS DyLight® 800, had similar recoveries when stained with Coomassie protein stain and therefore had similar amounts of conjugate present.

FIG. 2H is a bar graph showing the GAR-Dy 800 conjugate quantitation (fluorescence densitometry) of the gel of FIG. 2G and demonstrating background corrected volume of 34,309,695 for non-spiked 125 mM bicine (Lane 1); 37,231,174 for non-spiked 125 mM tricine (Lane 2); and 30,801,317 for non-spiked 75 mM sodium acetate (Lane 3).

FIG. 3A is a digital image of a fluorescent gel showing the recovery of isolated conjugatable rabbit IgG subsequently conjugated to NHS DyLight® 488 using non-spiked 125 mM bicine (Lane 1) and non-spiked 125 mM tricine (Lane 2); results demonstrate that bicine and tricine exhibited a desirable fluorescent signal for the rabbit IgG antibody-NHS DyLight® 488 conjugate.

FIG. 3B is a digital image of a fluorescent gel showing the recovery of isolated conjugatable mouse IgG subsequently conjugated to NHS DyLight® 680 using non-spiked 125 mM bicine (Lane 1) and non-spiked 125 mM tricine (Lane 2); results demonstrate that bicine and tricine exhibited a desirable fluorescent signal for the mouse IgG antibody-NHS DyLight® 680 conjugate.

FIG. 3C is a digital image of a fluorescent gel showing the recovery of isolated conjugatable mouse IgG1 subsequently conjugated to NHS DyLight® 405 using non-spiked 125 mM bicine (Lane 1) and non-spiked 125 mM tricine (Lane 2); results demonstrate that bicine and tricine exhibited a desirable fluorescent signal for the mouse IgG1 antibody-NHS DyLight® 405 conjugate.

FIG. 4A is a digital image of a fluorescent gel showing the recovery of isolated conjugatable PanRas 10, IgG2 A isotype subsequently conjugated to NHS Alexa Fluor® 647 using non-spiked 125 mM bicine (Lane 1) and non-spiked 125 mM tricine (Lane 2); results demonstrate that bicine and tricine exhibited a desirable fluorescent signal for the PanRas 10 primary antibody-Alexa Fluor® 647 conjugate.

FIG. 4B is a digital image of a fluorescent gel showing the recovery of isolated conjugatable STAT3, IgG2 A isotype subsequently conjugated to NHS DyLight® 800 using non-spiked 125 mM bicine (Lane 1) and non-spiked 125 mM tricine (Lane 2); results demonstrate that bicine and tricine exhibited a desirable fluorescent signal for the STAT3 primary antibody-NHS DyLight® 800 conjugate.

FIG. 4C is a digital image of a fluorescent gel showing the recovery of isolated conjugatable HDAC 1, rabbit polyclonal subsequently conjugated to NHS DyLight® 680 using non-spiked 125 mM bicine (Lane 1) and non-spiked 125 mM tricine (Lane 2); results demonstrate that bicine and tricine exhibited a desirable fluorescent signal for the HDAC primary antibody-NHS DyLight® 680 conjugate.

FIG. 5A is a digital image of a fluorescent gel showing the recovery of isolated conjugatable PanRas 10, IgG2 A isotype subsequently conjugated to NHS DyLight® 650 using non-spiked 125 mM bicine (Lane 1); results demonstrate that bicine and tricine exhibited a desirable fluorescent signal for the PanRas 10 primary antibody-NHS DyLight® 650 conjugate.

FIG. 5B is a digital image of a fluorescent gel showing the recovery of isolated conjugatable p 23, IgG1 isotype subsequently conjugated to NHS DyLight® 488 using non-spiked 125 mM bicine (Lane 1); results demonstrate that bicine and tricine exhibited a desirable fluorescent signal for the p 23 primary antibody-NHS DyLight® 488 conjugate.

FIG. 5C is a digital image of a fluorescent gel showing the recovery of isolated conjugatable STAT3, IgG2 A isotope subsequently conjugated to NHS DyLight® 650 using non-spiked 125 mM bicine (Lane 1); results demonstrate that bicine exhibited a desirable fluorescent signal for the STAT3 primary antibody-NHS DyLight® 650 conjugate.

FIG. 6 is a digital image of a fluorescent gel showing the recovery of GAR 650 (left), rabbit 650 IgG (middle), and mouse 650 IgG (right) conjugated to NHS DyLight® 650 using non-spiked 125 mM bicine (Lane 1); results demonstrate a desirable recovery of GAR 650, rabbit 650 IgG, and mouse 650 IgG conjugated with NHS DyLight® 650 using non-spiked 125 mM bicine.

FIG. 7A is a digital image of a fluorescent gel showing the recovery of isolated conjugatable GAR (0.5 mg/mL) subsequently conjugated to NHS DyLight® 650 using non-spiked 25 mM bicine (pH 9.2) (Lane 1A); 25 mM bicine (pH 9.2) spiked with 1 M borate (pH 9.4) (Lane 1B); non-spiked 50 mM bicine (pH 9.2) (Lane 2A); 50 mM bicine (pH 9.2) spiked with 1 M borate (pH 9.4) (Lane 2B); non-spiked 75 mM bicine (pH 9.2) (Lane 3A); 75 mM bicine (pH 9.2) spiked with 1 M borate (pH 9.4) (Lane 3B); non-spiked 75 mM sodium acetate (pH 7.0) (Lane 4A); and 75 mM sodium acetate (pH 7.0) spiked with 1 M borate (pH 9.4) (Lane 4B); results demonstrate that non-spiked bicine exhibited a desirable fluorescent signal for the GAR antibody-NHS DyLight® 650 conjugate unlike the non-spiked sodium acetate.

FIG. 7B is a bar graph showing the GAR-Dy 650 recovery (%) of the fluorescent gel of FIG. 7A and demonstrating a 78% recovery for non-spiked 25 mM bicine (pH 9.2) (Lane 1A); 86% recovery for 25 mM bicine spiked with 1 M borate (pH 9.4) (Lane 1B); 99% recovery for non-spiked 50 mM bicine (pH 9.2) (Lane 2A); 97% recovery for 50 mM bicine spiked with 1 M borate (pH 9.4) (Lane 2B); 88% recovery for non-spiked 75 mM bicine (pH 9.2) (Lane 3A); 99% recovery for 75 mM bicine spiked with 1 M borate (pH 9.4) (Lane 3B); 51% recovery for non-spiked 75 mM sodium acetate (pH 7.0) (Lane 4A); and 100% recovery for 75 mM sodium acetate spiked with 1 M borate (pH 9.4) (Lane 4B).

FIG. 8A is a digital image of a fluorescent gel (fluorescent scan) showing the recovery of isolated conjugatable rabbit IgG subsequently conjugated to NHS DyLight® 650 (Lane start “S” is the borate buffer exchanged using a 40 K resin subsequently conjugated with NHS Dy 650) from a sample comprising 1 mg/mL rabbit IgG and 10 mg/mL BSA (left) and from a sample comprising 0.1 mg/mL rabbit IgG and 10 mg/mL BSA (right); results demonstrate that bicine exhibited a desirable fluorescent signal for the rabbit IgG antibody-NHS DyLight® 650 conjugate.

FIG. 8B is a digital image of a Coomassie protein-stained gel showing the recovery of isolated conjugatable rabbit IgG subsequently conjugated to NHS DyLight® 650 (Lane start “S” is the borate buffer exchanged using a 40 K resin subsequently conjugated with NHS Dy 650) from a sample comprising 1 mg/mL rabbit IgG and 10 mg/mL BSA (left) and from a sample comprising 0.1 mg/mL rabbit IgG and 10 mg/mL BSA (right); results demonstrate that bicine exhibited desirable rabbit IgG antibody-NHS DyLight® 650 conjugate recovery.

FIG. 9 is a digital image of a fluorescent gel showing the recovery of isolated conjugatable RAS 10 subsequently conjugated to NHS DyLight® 650 from a sample comprising 1 mg/mL Ras 10, 50% gly (glycerol), 1 mg/mL BSA (left); isolated conjugatable p 23 subsequently conjugated to NHS DyLight® 488 (middle); isolated conjugatable STAT3 subsequently conjugated to NHS DyLight® 650 from a sample comprising 1 mg/mL STAT3 and 1 mg/mL BSA (right); results demonstrate that bicine produced isolated conjugatable primary antibodies RAS 10, p23, and STAT3 and subsequent conjugation produced a desirable fluorescent signal.

FIG. 10 is a digital image of a fluorescent gel showing the recovery of isolated conjugatable human IgG from human serum using a matrix comprising (i) a porous size exclusion support and (ii) one or more cationic moieties associated with the porous size exclusion support, wherein the isolated conjugatable human IgG was subsequently conjugated to NHS DyLight® 650, and wherein the matrix was equilibrated with 5 mM bicine, 80 mM NaCl, pH 9.0 (Lanes 1 and 2 are duplicate isolated samples from human serum; Lane 3 is the human serum starting material); results demonstrate that the matrix produced isolated conjugatable human IgG and subsequent conjugation produced a desirable fluorescent signal.

FIG. 11A is a digital image of a fluorescent gel showing the recovery of isolated conjugatable Goat Anti-Rabbit from an antibody-BSA mixture using a matrix comprising (i) a porous size exclusion support and (ii) one or more cationic moieties associated with the porous size exclusion support, wherein the isolated conjugatable Goat Anti-Rabbit was subsequently conjugated to DyLight® 488, and wherein the matrix was equilibrated with 125 mM bicine, 100 mM NaCl, pH 9.0 (Lanes 1 and 2 are duplicate samples purified from the antibody-BSA mixture using the matrix; Lane 3 is the antibody-BSA mixture prior to cleanup; and Lane 4 is a control antibody sample without BSA); results demonstrate that the matrix produced isolated conjugatable Goat Anti-Rabbit and subsequent conjugation produced a desirable fluorescent signal.

FIG. 11B is a digital image of a fluorescent gel showing the recovery of isolated conjugatable Rat IgG from an antibody-BSA mixture using a matrix comprising (i) a porous size exclusion support and (ii) one or more cationic moieties associated with the porous size exclusion support, wherein the isolated conjugatable Rat IgG was subsequently conjugated to DyLight® 800, and wherein the matrix was equilibrated with 125 mM bicine, 100 mM NaCl, pH 9.0 (Lanes 1 and 2 are duplicate samples purified from the antibody-BSA mixture using the matrix; Lane 3 is the antibody-BSA mixture prior to cleanup; and Lane 4 is a control antibody sample without BSA); results demonstrate that the matrix produced isolated conjugatable Rat IgG and subsequent conjugation produced a desirable fluorescent signal.

FIG. 11C is a digital image of a fluorescent gel showing the recovery of isolated conjugatable Mouse IgG from an antibody-BSA mixture using a matrix comprising (i) a porous size exclusion support and (ii) one or more cationic moieties associated with the porous size exclusion support, wherein the isolated conjugatable Mouse IgG was subsequently conjugated to DyLight® 650, and wherein the matrix was equilibrated with 125 mM bicine, 100 mM NaCl, pH 9.0 (Lanes 1 and 2 are duplicate samples purified from the antibody-BSA mixture using the matrix; Lane 3 is the antibody-BSA mixture prior to cleanup; and Lane 4 is a control antibody sample without BSA); results demonstrate that the matrix produced isolated conjugatable Mouse IgG and subsequent conjugation produced a desirable fluorescent signal.

### DETAILED DESCRIPTION

### I. Abbreviations

BSA: Bovine Serum Albumin
DEED: Diethylethylenediamine
Epi: Epichlorohydrin
HEC: hydroxyethyl cellulose
HEPES: 4-(2-hydroxyethyl)-1-piperazineethanesulfonic acid
IgG: Immunoglobulin G
kDa: Kilodalton
MWCO: Molecular weight cut-off
PDA: Diaminopentane
PEI: Branched polyethylene imine
TRIS: Tris (hydroxymethyl)aminomethane

### II. Overview of Terms, Ranges, and Definitions

Both the foregoing general description and the following detailed description are exemplary and explanatory only and are not intended to limit the scope of the present disclosure.

As used herein, the use of the singular includes the plural unless specifically stated otherwise. For example, the singular forms “a”, “an” and “the” as used in the specification also include plural aspects unless the context dictates otherwise. Similarly, any singular term used in the specification also means plural or vice versa, unless the context dictates otherwise.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing examples from discussed prior art, the example numbers are not approximates unless the word “about” is recited. Furthermore, not all alternatives recited herein are equivalents.

The term “or combinations thereof” as used herein refers to all permutations and combinations of the listed items preceding the term. For example, “A, B, C, or combinations thereof” is intended to include at least: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth.

The following terms and definitions are provided:

Acyl Halide: A chemical functional group —C(O)X, wherein X is a halogen, such as Br, F, I, or Cl.

Alcohol: A chemical functional group including at least one hydroxyl group. Alcohols may be monohydric (including one-OH group), dihydric (including two-OH groups; including diols, such as glycols), trihydric (including three-OH groups; including triols, such as glycerol), or polyhydric (including four or more-OH groups; including polyols). The organic portion of the alcohol may be aliphatic, cycloaliphatic (alicyclic), heteroaliphatic, cycloheteroaliphatic (heterocyclic), polycyclic, aryl, or heteroaryl, and may be substituted or unsubstituted. Alcohol groups are examples of a heteroaliphatic group.

Aldehyde: A carbonyl-bearing functional group having a formula

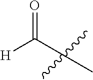 [SMILES: *C=O]

where the wavy line indicates that the functional group can be attached to any other moiety, but that such moiety simply is not shown.

Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a C═C double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkoxy: A radical (or substituent) having the structure-OR, where R is a substituted or unsubstituted aliphatic group.

Alkyl: A hydrocarbon group having a saturated carbon chain. The chain may be cyclic, branched, or unbranched.

Alkynyl: A hydrocarbon group having at least one carbon-carbon triple bond. An alkynyl group can be branched, straight-chain, or cyclic (e.g., cycloalkynyl).

Amide: A chemical functional group —C(O) N(R′) (R″) where R′ and R″ independently hydrogen, aliphatic, heteroaliphatic, haloaliphatic, aryl (such as optionally substituted phenyl or benzyl), heteroaryl, haloaryl, alkylsulfano, or other functionality.

Amino: A chemical functional group —N(R) R′ where R and R′ are independently hydrogen, aliphatic, heteroaliphatic, haloaliphatic, aryl (such as optionally substituted phenyl or benzyl), heteroaryl, haloaryl, alkylsulfano, or other functionality.

Antibody: Immunoglobulins or immunoglobulin-like molecules (including by way of example and without limitation, IgA (Immunoglobulin A), IgD (Immunoglobulin D), IgE (Immunoglobulin E), IgG (Immunoglobulin E) and IgM (Immunoglobulin M), combinations thereof, and similar molecules produced during an immune response in any chordate such as a vertebrate, for example, in mammals such as humans, goats, rabbits and mice) and fragments thereof that specifically bind to a molecule of interest (or a group of highly similar molecules of interest) to the substantial exclusion of binding to other molecules. An “antibody” typically comprises a polypeptide ligand having at least a light chain or heavy chain immunoglobulin variable region that specifically recognizes and binds an epitope of an antigen. Immunoglobulins are composed of a heavy and a light chain, each of which has a variable region, termed the variable heavy (VH) region and the variable light (VL) region. Together, the VH region and the VL region are responsible for binding the antigen recognized by the immunoglobulin. Exemplary immunoglobulin fragments include, without limitation, proteolytic immunoglobulin fragments [such as F(ab′) 2 fragments, Fab′ fragments, Fab′-SH fragments and Fab fragments as are known in the art], recombinant immunoglobulin fragments (such as sFv fragments, dsFv fragments, bispecific sFv fragments, bispecific dsFv fragments, ‘F(ab)’₂ fragments, single chain Fv proteins (“scFv”), and disulfide stabilized Fv proteins (“dsFv”). Other examples of antibodies include diabodies, and triabodies (as are known in the art), and camelid antibodies. “Antibody” also includes genetically engineered molecules, such as chimeric antibodies (for example, humanized murine antibodies), and heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

Aromatic: A cyclic or conjugated group comprising, unless specified otherwise, from 5 to 15 ring atoms having at least a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

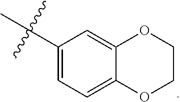 [SMILES: *c1ccc2c(c1)OCCO2]

However, in certain examples, context or express disclosure may indicate that the point of attachment is through a non-aromatic portion of the condensed ring system. For example,

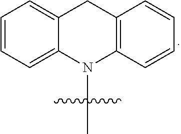 [SMILES: *N1c2ccccc2Cc2ccccc21]

An aromatic group may comprise only carbon atoms in the ring, such as in an aryl group, or it may comprise one or more ring carbon atoms and one or more ring heteroatoms comprising a lone pair of electrons (e.g. S, O, N, P, or Si), such as in a heteroaryl group. Aromatic groups may be substituted with one or more groups other than hydrogen, such as aliphatic, heteroaliphatic, haloaliphatic, aryl (such as optionally substituted phenyl or benzyl), heteroaryl, haloaryl, alkylsulfano, or other functionality, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, aryl (such as optionally substituted phenyl or benzyl), heteroaryl, haloaryl, alkylsulfano, or other functionality.

Biological Sample: Hematological, cytological and histological specimens, such as cells, cell cultures, hybridomas, single-celled organisms (e.g. yeast and bacteria), 3D cell cultures (e.g. spheroids and organoids), tissues, whole organisms (e.g. flies or worms), cell-free extracts, or a fluid sample comprising any biological matter (e.g., blood, serum, plasma, sputum, urine, cerebrospinal fluid). Biological samples can be from a plant or animal (e.g., human, mouse, fly, worm, fish, frog, fungi, and the like). A biological sample can refer to a sample that has been processed by filtration and/or centrifugation and can include supernatants of cell cultures and homogenized tissue or broken up cells.

Carbamate: A chemical functional group —OC(O)NRR′, wherein R and R′ independently are hydrogen, aliphatic, heteroaliphatic, haloaliphatic, aryl (such as optionally substituted phenyl or benzyl), heteroaryl, haloaryl, alkylsulfano, or other functionality.

Carbonate: A chemical functional group with the formula —OCO₂R where R is hydrogen, aliphatic, heteroaliphatic, haloaliphatic, aryl (such as optionally substituted phenyl or benzyl), heteroaryl, haloaryl, alkylsulfano, or other functionality.

Carboxamide: A chemical functional group —N(R)acyl, or —C(O)amino, where R is hydrogen, aliphatic, heteroaliphatic, haloaliphatic, aryl (such as optionally substituted phenyl or benzyl), heteroaryl, haloaryl, alkylsulfano, or other functionality.

Carboxylic Acid: A chemical functional group —C(O)OH. An anion of a carboxylic acid group has a formula —C(O)O⁻ wherein the negatively charged oxygen can be balanced with a positively charged ion.

Conjugated: A molecule being attached to another molecule, either directly or indirectly, either by covalent or noncovalent linkage.

Conjugatable: A large molecule, as described herein, is “conjugatable” in the sense that it can undergo conjugation with a label after having been exposed to an amine buffer according to the present disclosure and without necessitating buffer exchange of the amine buffer or buffer spiking steps.

Cyano: A chemical functional group —CN.

Disulfide: A chemical functional group —SSR^{a}, wherein R^{a} is selected from hydrogen, aliphatic, heteroaliphatic, haloaliphatic, aryl (such as optionally substituted phenyl or benzyl), heteroaryl, haloaryl, alkylsulfano, or other functionality.

Epoxide: A chemical functional group having a cyclic ether with a 3-membered ring having a general formula,

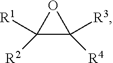

where R¹-R⁴ independently are hydrogen, aliphatic, heteroaliphatic, haloaliphatic, aryl (such as optionally substituted phenyl or benzyl), heteroaryl, haloaryl, alkylsulfano, or other functionality.

Ester: A chemical functional group

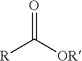

where R and R′ are independently aliphatic, heteroaliphatic, haloaliphatic, aryl (such as optionally substituted phenyl or benzyl), heteroaryl, haloaryl, alkylsulfano, or other functionality.

Ether: A chemical functional group R—O—R′, where R and R′ are independently aliphatic, heteroaliphatic, haloaliphatic, aryl (such as optionally substituted phenyl or benzyl), heteroaryl, haloaryl, alkylsulfano, or other functionality.

Fluorescent Dye: A type of label that is molecule comprising a functional group or portion of a compound that causes the compound (or a sample or composition comprising the compound), to fluoresce. In some aspects, the fluorophore can fluoresce when the compound (or a sample or composition comprising the compound) is exposed to an excitation source or after being cleaved from a compound to which the fluorophore is conjugated.

Functional Group: A group of atoms within a molecule that is responsible for the characteristic chemical reactions of the molecule. Exemplary functional groups include, without limitation, alkyl, alkenyl, alkynyl, aryl, halo (fluoro, chloro, bromo, iodo), epoxide, hydroxyl, carbonyl (ketone), aldehyde, carbonate ester, carboxylate, carboxyl, ether, ester, peroxy, hydroperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

Halo: refers to fluoro, chloro, bromo, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroaryl: An aryl group comprising at least one heteroatom, which can be selected from, but not limited to, oxygen, nitrogen, sulfur, silicon, boron, selenium, phosphorous, and oxidized forms thereof within the ring. Heteroaryl groups can comprise a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, aryl (such as optionally substituted phenyl or benzyl), heteroaryl, haloaryl, alkylsulfano, or other functionality. In some aspects, a fluorophore can also be described herein as a heteroaryl group.

### Hydroxyl: Refers to —OH.

Imine: An organic compound containing a —C═NR group, where R is hydrogen, aliphatic, heteroaliphatic, haloaliphatic, aryl (such as optionally substituted phenyl or benzyl), heteroaryl, haloaryl, alkylsulfano, or other functionality.

Immunoglobin G (IgG): One of the primary classes of immunoglobins having heavy chains known as gamma-chains.

Isoelectric Point (pI): The pH at which a molecule carries no net electric charge. Polymeric molecules, such as proteins comprising of amino acids, can be positive, neutral, negative, or polar in nature, giving the polymeric material an overall charge. A molecule with a low pI value carries a net negative charge at neutral pH, and a molecule with a high pI value carries a net positive charge at neutral pH.

Molecular Weight Cut-Off or “MWCO”: The lowest molecular weight of sample components that are excluded by the matrix, such as the smaller sample components. For example, one or more small molecules, such as, but not limited to BSA, are excluded by the matrix, whereas the larger sample components, such as one or more large molecules, elute faster, and are recovered.

Multimodal: The ability of a material or compound, such as a resin or matrix, to provide plural different types of interactions between the resin or matrix and a desired molecule to contribute to the separation of a first desired molecule from a second molecule, such as by retention of the first and/or second molecule to the resin or matrix.

Negatively Charged Small Molecule: A molecule with a low isoelectric point (pI), which carries a net negative charge at a neutral pH.

Phosphate: A chemical functional group —O—P(O)(OR^{a})₂, wherein each R^{a} independently is hydrogen, aliphatic, heteroaliphatic, haloaliphatic, aryl (such as optionally substituted phenyl or benzyl), heteroaryl, haloaryl, alkylsulfano, or other organic functional group.

Positively Charged Large Molecule: A molecule with a high isoelectric point (pI), which carries a net positive charge at a neutral pH.

Sample: Any fluid or solution comprising at least two molecules that need to be separated, wherein a first of the at least two molecules is a small molecule, as defined herein, and a second of the at least two molecules is a large molecule, as defined herein. In some aspects, the sample may comprise a small molecule, such as but not limited to a stabilizing molecule. The stabilizing molecule can be a purified protein isolated from natural or recombinant sources. For example, a recombinant albumin, a native albumin, human serum albumin, an albumin-like stabilizer, bovine serum albumin, comparable mammalian serum (such as but not limited to, rabbit serum and mouse serum), ovalbumin, glycerol, and/or gelatin. A sample can also include one or more molecules derived from biological samples.

Size Exclusion Support: An inert porous solid that has a porosity which determines the size of a molecule that may be included or excluded from entering the pores. In some disclosed aspects, the pores of a porous size exclusion support have a molecular size cut-off (MWCO) of 40 kDa or greater than 40 kDa.

Sulfonamide: A chemical functional group —SO₂amil or —N(R)sulfonyl, where R is hydrogen, aliphatic, heteroaliphatic, haloaliphatic, aryl (such as optionally substituted phenyl or benzyl), heteroaryl, haloaryl, alkylsulfano, or other functionality.

Sulfonate: A chemical functional group —SO₃⁻, wherein the negative charge of the sulfonate group may be balanced with a positive counterion, such as an M⁺ counter ion, wherein M⁺ may be an alkali ion, such as K⁺, Na⁺, Li⁺; an ammonium ion, such as ⁺N(R^{b})₄, where R^{b} is hydrogen, aliphatic, heteroaliphatic, haloaliphatic, aryl (such as optionally substituted phenyl or benzyl), heteroaryl, haloaryl, alkylsulfano, or other functionality; or an alkaline earth ion, such as [Ca²⁺]_{0.5}, [Mg²⁺]_{0.5}, or [Ba²⁺]_{0.5}.

Sulfonyl: A chemical functional group with the general formula:

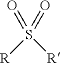

where R represents the rest of the molecule to which the sulfonyl group is bound and R′ is selected from hydrogen, aliphatic, heteroaliphatic, haloaliphatic, aryl (such as optionally substituted phenyl or benzyl), heteroaryl, haloaryl, alkylsulfano, or other functionality.

Thioester: A chemical functional group with the general formula:

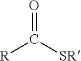

where R represents the rest of the molecule to which the thioester group is bound and R′ is selected from aliphatic, heteroaliphatic, haloaliphatic, aryl (such as optionally substituted phenyl or benzyl), heteroaryl, haloaryl, alkylsulfano, or other functionality.

Thioether: A chemical functional group R—S—R′ where R and R′ independently are selected from aliphatic, heteroaliphatic, haloaliphatic, aryl (such as optionally substituted phenyl or benzyl), heteroaryl, haloaryl, alkylsulfano, or other functionality. A thioether is similar to an ether, except that a thioether contains a sulfur atom in place of the oxygen atom of an ether.

The terms “separation,” “extraction,” “extracted,” “removal,” “reducing” or “reducing the quantity of,” or “purification” generally refer to removing or isolating a substance, e.g., a small molecule, such as BSA, or a large molecule or a biomolecule, such as IgG, from a mixture comprising the small molecule and/or large molecule. An extracted substance or a sample from which a substance has been extracted has significantly decreased quantities of components that were present in the sample prior to separation, and the extracted substance can be substantially reduced, substantially removed, substantially concentrated, substantially pure, or pure (devoid of any contaminants), compared to prior to being extracted.

The present disclosure uses the terms “small molecule” and “large molecule” which refer to species to be separated from one another. “Small molecule” or “smaller molecule” generally refers to any molecule having a molecular weight of less than 100 kDa, and a “large molecule” is a molecule having a molecular weight of greater than or equal to 100 kDa. The small molecule (such as but not limited to a biomolecule) may be being used to treat, derivatize, conjugate, cross-link, label, tag, chemically or biologically modify the large molecule for further analysis. The small molecule can be a stabilizing molecule, such as but not limited to, a purified protein isolated from natural or recombinant sources. For example, a recombinant albumin, a native albumin, human serum albumin, an albumin-like stabilizer, bovine serum albumin, comparable mammalian serum, ovalbumin and/or gelatin. Derivatization includes labeling molecules with labels such as dyes, affinity tags, radioactive labels, mass tags, metals, and the like. Derivatization also includes chemically modifying molecules by reduction, oxidization, methylation, biologically or biochemically modifying biomolecules, etc. Derivatives of biomolecules include, again without limitation: tagged proteins or nucleic acids; labeled biomolecules that are labeled with a variety of labels such as but not limited to dyes, fluorescent dyes, radioactive labels, affinity labels, mass-tags, metals, etc.; conjugated biomolecules, including conjugated antibodies; biomolecules conjugated to nanoparticles; metals, such as gold conjugated to nanoparticles; dyes or labels, such as biotin conjugated to toxins; chemical derivatives of biomolecules, such as but not limited to, reduced proteins, oxidized proteins, methylated nucleic acids, and proteins with sulfhydryl modified proteins. Large molecules and/or biomolecules include, for example and without limitation, proteins, glycoproteins, and antibodies. In one example, the small molecule is BSA having a molecular weight of 67,000 Da. In one example, the large molecule is IgG having a molecular weight of 150 kDa. In certain disclosed examples, a sample comprises a mixture of BSA and IgG.

All literature and similar materials cited in this application including, but not limited to, patents, patent applications, articles, books, treatises, and internet web pages, regardless of the format of such literature and similar materials, are expressly incorporated by reference in their entirety for any purpose. If one or more of the incorporated literature and similar materials defines or uses a term in such a way that it contradicts that term's definition in this application, the definitions provided by this specification control. While the present teachings are described in conjunction with various aspects, it is not intended that the present teachings be limited to such aspects. On the contrary, the present teachings encompass various alternatives, modifications, and equivalents, as will be appreciated by those in the art with the benefit of the present disclosure.

### III. Introduction

BSA, a protein derived from bovine blood plasma, is commonly used at 10 mg/mL as a stabilizing agent in antibody samples, where antibody concentrations are typically <1 mg/mL. However, BSA interferes with antibody labeling, reducing the labeling efficiency. To address this, BSA is removed from the target antibody prior to labeling. Conventional methods and kits for BSA removal are ineffective due to low antibody recovery rates and/or insufficient BSA removal efficiency.

High-capacity mixed-mode resins, combining ion-exchange and size exclusion components, enables the efficient separation of high concentrations of BSA from one or more antibodies in a single step. However, purified antibodies are often used for downstream applications such as conjugating fluorescent dyes for Western blotting, immunofluorescence, and imaging. This conjugation process typically involves intermediate sample preparation steps, including buffer exchange (e.g., phosphate-buffered saline or borate) or spiking with amine-free buffers like carbonate or borate buffers. These intermediate steps can result in sample loss or dilution, ultimately reducing conjugation efficiency.

Disclosed herein is a method for removing small molecules, like BSA, using a buffer that is suitable for use in both a small molecule-removal step and subsequent labeling of large molecules isolated from the small molecules. As such, the method facilitates removing intermediate sample preparation steps, thereby reducing sample loss and sample dilution with desirable conjugation efficiency. Also disclosed herein is a composition comprising an amine buffer and a matrix comprising (i) a porous size exclusion support, and (ii) one or more cationic moieties associated with the porous size exclusion support for use in the method. A kit for isolating conjugated antibodies is also disclosed herein.

### IV. Methods

Certain disclosed aspects concern a method, comprising combining an amine buffer with a matrix comprising (i) a porous size exclusion support, and (ii) one or more cationic moieties associated with the porous size exclusion support, wherein the matrix can separate one or more molecules in a sample by using a molecular weight of the one or more molecules, a charge of the one or more molecules, an isoelectric point (pI) of the one or more molecules, or a combination thereof. In aspects disclosed herein, the amine buffer is an equilibration and/or conjugation buffer that allows for the conjugation of labels to the one or more large molecules in the sample.

In aspects disclosed herein, the amine buffer can equilibrate the matrix and enable conjugatable molecules in a sample. In aspects disclosed herein, the amine buffer has a pH ranging from 7.5 to 9.5, such as 7.5 to 9.4, or 8.0 to 9.5, or 7.5 to 9.4, or 9.0 to 9.5, or 9.0 to 9.4.

Aspects of the present disclosure describe matrixes for isolating one or more large molecules. In some aspects, the large molecules can be, but are not limited to, a glycoprotein, phosphoprotein, antibody, or immunoglobulin. In certain aspects, the large molecules are conjugatable large molecules such as, but not limited to, conjugatable antibodies.

In certain aspects, the method can further comprise adding one or more labels to the conjugatable large molecule to directly or indirectly produce a detectable signal. In aspects disclosed herein, the labels can include, but are not limited to, dyes, affinity tags, radioactive labels, mass tags, metals, and the like. In some aspects, the dyes comprise one or more reactive groups such as, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, amines, ureas, pseudoureas, semicarbazides, carbodiimides, carbamates, imines, azides, alkynes (including strained alkynes, such as DIBO and DBCO), azo compounds, azoxy compounds, and nitroso compounds. Reactive functional groups also include those used to prepare bioconjugates, e.g., N-hydroxysuccinimide esters (or succinimidyl esters (SE)), maleimides, sulfodichlorophenyl (SDP) esters, sulfotetrafluorophenyl (STP) esters, tetrafluorophenyl (TFP) esters, pentafluorophenyl (PFP) esters, nitrilotriacetic acids (NTA), aminodextrans, cyclooctyneamines and the like.

In some aspects, the dye can be a fluorescent dye such as, but not limited to, a rhodamine- and cyanine-based fluorescent dyes. Exemplary non-limiting fluorescent dyes can include those available from Thermo Fisher Scientific (e.g., Alexa Fluor®405, Alexa Fluor®488, Alexa Fluor®546, Alexa Fluor®555, Alexa Fluor®568, Alexa Fluor®594, Alexa Fluor®647, Alexa Fluor®680, Alexa Fluor®700, DyLight® 405, DyLight® 488, DyLight® 550, DyLight® 650) and ATTO-TEC GmbH, Germany (e.g., ATTO 488 and ATT0594).

In some aspects, the fluorescent dye(s) are conjugated to one or more conjugatable large molecules such as, but not limited to, one or more conjugatable antibodies to directly or indirectly produce a detectable signal. In certain aspects, the detectable signal is a fluorescence signal, which is typically a change in fluorescence, such as a change in the intensity, excitation or emission wavelength, distribution of fluorescence, fluorescence lifetime, fluorescence polarization, or a combination thereof. In such aspects of the method, the conjugation step can be performed without having to spike the composition with a buffer, and/or exchange the amine buffer with a conjugation buffer.

Also disclosed herein is a method, comprising exposing a sample to a matrix and an amine buffer. In some aspects, the matrix and amine buffer are as described herein. In certain aspects, a sample comprising at least one small molecule and at least one large molecule is exposed to the matrix, wherein the large molecule elutes faster than the small molecule that is trapped by the matrix. In particular aspects, the sample is first exposed to the matrix and then exposed to the amine buffer. In other aspects, the sample is first exposed to the amine buffer and then exposed to the matrix.

Samples that may be processed by methods of the disclosure may be any type of biological or clinical sample comprising biomolecules or derivatives thereof from which small molecules are to be separated or removed. In aspects disclosed herein, the sample comprises one or more biomolecules. In aspects disclosed herein, the biomolecule can be an antibody (e.g., a primary antibody or a secondary antibody). In some aspects, the biomolecule is an antibody fragment. In aspects disclosed herein, the antibody is polyclonal, monoclonal or engineered and may be from any source (e.g., chicken or a mammal, such as a llama, human mouse, rabbit, goat, rat, etc.). In certain aspects, the sample may comprise humanized antibodies. Some exemplary non-limiting samples include samples comprising the small molecule BSA and the large molecule IgG.

In some aspects, the porous size exclusion support comprising one or more cationic moieties bind to, or associate with, a small molecule. In some aspects, small molecules may constitute a sample impurity or contaminant. In some aspects, biomolecules such as, but not limited to, antibodies, are separated by removing the small molecules, such as, but not limited to, BSA, in one step. In particular aspects, the method further comprises isolating the large molecule. In certain aspects, the isolated large molecule(s), amine buffer, or a combination thereof, are collected as flow-through.

In certain aspects, the method further comprises adding a dye disclosed herein to conjugate with the isolated antibody (or antibodies) to directly or indirectly produce a detectable signal. In some aspects, fluorescent dyes can be conjugated to large molecules such as, but not limited to, antibodies, to generate detectable signals, typically fluorescence changes (e.g., intensity, wavelength, lifetime, polarization). In such aspects of the method, the conjugation step can be performed without having to spike the composition with a buffer, and/or exchange the amine buffer with a conjugation buffer.

In aspects disclosed herein, the sample is exposed to a matrix and an amine buffer in a container. In certain aspects, the container is a columnar container, a tube, a multi-well tube, a multi-well plate or a multi-well filter plate. Exemplary containers include, but are not limited to, a test tube, a spin column, a multi-well plate, a multi-well filter plate, a micro-well plate, or a micro-well filter plate.

In some aspects, the method further comprises subjecting the matrix, amine buffer, and sample to gravity flow, a centrifugal force, a positive pressure, a negative pressure, a vacuum, or a combination thereof. Structures that allow applications of the above-mentioned pressures or forces include, without limitation: a syringe that can be drawn to cause a positive pressure; a vacuum frit for generating negative pressure; and/or tubes or containers adaptable to commercially available centrifuges or rotatory devices.

In certain aspects, the method can further comprise collecting the flow-through in a receptacle. In particular aspects, the receptacle is attached to the container. In some aspects, the receptacle is detachable from the container and can be collected by a user such that the contents of a receptacle can be used or removed by a user as desired. In some aspects, the receptacle is a wash plate or a collection plate. In some aspects, the receptacle collects one or more large molecule, such as, but not limited to, antibodies with substantially reduced small molecule levels or wholly absent of any small molecules.

Methods of the disclosure advantageously reduce the time required for processing a sample, and/or increase the quantity of small molecules removed from the sample.

### V. Composition

Certain disclosed aspects of the present disclosure concern a composition comprising (i) a matrix for separating large and small molecules, such as biomolecules and BSA, from each other; and (ii) an amine buffer. In some aspects, molecules are separated from each other using differences in one or more properties such as the size of the molecules, charge of the molecules, the isoelectric point (pI) of the molecules, and/or a combination of these properties.

In certain aspects, molecules can be separated from each other based on one or more separation matrix properties such charge on the matrix and size exclusion properties of the matrix. In aspects disclosed herein, the amine buffer equilibrates the matrix and/or prepares the biomolecules in a sample for conjugation thereby providing a conjugatable biomolecule.

In some aspects, matrixes for separating, extracting, removing, and/or reducing the quantity of small molecules from large molecules are disclosed. In some aspects, the small molecule can be, but is not limited to, a protein, globular protein, Sphero protein, serum albumin protein, polypeptide, and/or the like.

In certain aspects, the small molecules can be separated from larger molecules using one or more properties, including, but not limited to, the isoelectric point of the molecules. In some aspects, the small molecules can be negatively charged small molecules. In particular aspects disclosed herein, the negatively charged small molecules can have an isoelectric point (pI) value in the range of 4.5-5.5. In some aspects, the negatively charged small molecules may have a pI value of 4.5, 4.6, 4.7 4.8, 4.9, 5.0, 5.1, 5.2, 5.3, 5.4, or 5.5. In one exemplary non-limiting aspect, the negatively charged small molecule has a pI value of 4.9.

In some aspects, the matrix separates, extracts, removes, and/or reduces the quantity of small molecules from large molecules based on, but not limited to, the size of the molecules. In some aspects of the present disclosure, the small molecules can have a molecular weight that is less than 100 kDa, less than 80 kDa, and/or less than 70 kDa. In some aspects, the small molecules may have a molecular weight of from 50 kDa to 80 kDa, such as 50 kDa, 51 kDa, 52 kDa, 53 kDa, 54 kDa, 55 kDa, 56 kDa, 57 kDa, 58 kDa, 59 kDa, 60 kDa, 61 kDa, 62 kDa, 63 kDa, 64 kDa, 65 kDa, 66 kDa, 65 kDa, 66 kDa, 67 kDa, 68 kDa, 69 kDa, 70 kDa, 71 kDa, 72 kDa, 73 kDa, 74 kDa, 75 kDa, 76 kDa, 77 kDa, 78 kDa, 79 kDa, and/or 80 kDa. In some exemplary non-limiting aspects, the small molecule is BSA with a molecular weight of 67,000 Da (67 kDa).

In certain aspects, the small molecules can be separated from larger molecules using one or more properties including, but not limited to, the isoelectric point of the molecules. In some aspects, the large molecules can be positively charged large molecules. In particular disclosed aspects, the positively charged large molecules may have an isoelectric point (pI) value in the range of 8.0-11.5. In some aspects, the positively charged large molecules may have a pI value of 8.0, 8.1, 8.2, 8.3, 8.4, 8.5, 8.6, 8.7 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, and/or 11.5. In one exemplary non-limiting aspect, the positively charged large molecule has a pI value of 11.0.

In some aspects, the matrix separates, extracts, removes, and/or reduces the quantity of small molecules from large molecules based on, but not limited to, the size of the molecules. In some aspects, the large molecules may have a molecular weight greater than or equal to 100 kDa, and in certain aspects a molecular weight greater than or equal to 150 kDa. For example, 100 kDa, 101 kDa, 102 kDa, 103 kDa, 104 kDa, 105 kDa, 106 kDa, 107 kDa, 108 kDa, 109 kDa, 110 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, 140 kDa, 141 kDa, 142 kDa, 143 kDa, 144 kDa, 145 kDa, 146 kDa, 147 kDa, 148 kDa, 149 kDa, 150 kDa, 151 kDa, 152 kDa, 153 kDa, 154 kDa, 155 kDa, 156 kDa, 157 kDa, 158 kDa, 159 kDa, 160 kDa, 161 kDa, 162 kDa, 163 kDa, 164 kDa, 165 kDa, 166 kDa, 167 kDa, 168 kDa, 169 kDa, 170 kDa, 180 kDa, 220 kDa, 250 kDa, 300 kDa, 350 kDa, 450 kDa, 550 kDa, 750 kDa, 900 kDa. In one exemplary non-limiting aspect, the large molecule is IgG having a molecular weight of 150,000 Da (150 kDa).

### A. Amine Buffer

The composition of the present disclosure also comprises an amine buffer. The amine buffer facilitates increasing the binding capacity of the matrix to smaller sample components, decreasing the binding capacity of the matrix to larger sample components, and producing one or more conjugatable molecules from the larger sample components. For example, and without being bound by a single theory of operation, it currently is believed that the amine buffer increases the binding capacity of the cationic moiety to a small molecule, such as BSA, that is present in a sample. It also is believed that the amine buffer facilitates decreasing the binding capacity of the cationic moiety to a large molecule, such as an antibody, that is present in the sample with the small molecule. Further, it is believed that the amine buffer can facilitate preparing the large molecule for conjugation by making it conjugatable to one or more labels.

As such, the amine buffer facilitates a method wherein, after removing the small molecules in the presence of the amine buffer, a label (e.g., a dye) can be provided for conjugating with the recovered large molecules without having to perform any buffer spiking and/or buffer exchange.

In some aspects, the amine buffer has a structure according to Formula I,

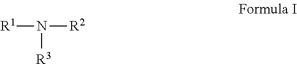

wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof.

In particular aspects, the amine buffer has a structure according to Formula I, wherein each of R¹, R², and R³ independently is selected from hydrogen or an aliphatic group, wherein the aliphatic group is alkyl, alkenyl, alkynyl, provided that at least one of R¹, R², and R³ comprises a carboxylic acid or an anion thereof. In some aspects, R¹, R², and R³ independently is selected from hydrogen or alkyl, wherein the alkyl group is methyl, ethyl, propyl, butyl, pentyl, hexyl, heptyl, octyl, nonyl, and decyl, provided that at least one of R¹, R², and R³ comprises a carboxylic acid or an anion thereof. In particular aspects, each of R¹, R², and R³ independently is selected from hydrogen or an alkenyl group or an alkynyl group, provided that at least one of R¹, R², and R³ comprises a carboxylic acid or an anion thereof.

In some aspects, the amine buffer has a structure according to Formula I, wherein each of R¹, R², and R³ independently is selected from hydrogen or a heteroaliphatic group, wherein the heteroaliphatic group comprises a heteroatom (e.g., oxygen, sulfur, nitrogen, phosphorus, or the like), provided that at least one of R¹, R², and R³ comprises a carboxylic acid or an anion thereof. In certain aspects, the heteroaliphatic group is heteroalkyl, heteroalkenyl, heteroalkynyl. In certain aspects, each of R¹, R², and R³ independently is selected from hydrogen or a heteroalkyl group, wherein the heteroalkyl group comprises an alcohol, an alkoxy group, an amino group, a thioether group, or a thiol group, provided that at least one of R¹, R², and R³ comprises a carboxylic acid or an anion thereof. In particular aspects, the R¹, R², and R³ groups independently are selected from hydrogen or a heteroalkenyl group or a heteroalkynyl group, provided that at least one of R¹, R², and R³ comprises a carboxylic acid or an anion thereof.

In certain aspects the amine buffer is a secondary amine. In particular aspects, the amine buffer is a secondary amine, R¹—NH—R², where one of R¹ and R² comprises a heteroaliphatic group, and the other comprises a carboxylic acid or anion thereof (e.g., —(CH₂)ₙCOOH, wherein n is an integer ranging from 1 to 20, such as 1 to 15, or 1 to 10, or 1 to 5). In some aspects, the heteroaliphatic group is monohydric (having one —OH group), dihydric (having two —OH groups), trihydric (having three —OH groups), or polyhydric (having four or more —OH groups). In some aspects, the heteroaliphatic group is —C[(CH₂)ₘOH]₃ wherein m is an integer ranging from 1 to 20, such as 1 to 15, or 1 to 10, or 1 to 5. In some aspects, the carboxylic acid group is —CH₂COOH. In a non-limiting exemplary aspect, the amine buffer is tricine.

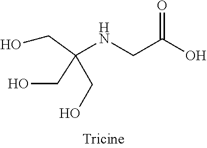 [SMILES: O=C(O)CNC(CO)(CO)CO]

In some aspects, the amine buffer is a tertiary amine. In certain aspects, the amine buffer is a tertiary amine according to Formula I, wherein two of R¹, R², and R³ independently are a heteroaliphatic group and the remaining of R¹, R², and R³ comprises a carboxylic acid or anion thereof (e.g., —(CH₂)ₙCOOH, wherein n is an integer ranging from 1 to 20, such as 1 to 15, or 1 to 10, or 1 to 5). In particular aspects, the heteroaliphatic group is monohydric (having one —OH group), dihydric (having two —OH groups), trihydric (having three —OH groups), or polyhydric (having four or more —OH groups). In some aspects, each heteroaliphatic group independently is selected from —(CH₂)ₘOH wherein m is an integer ranging from 1 to 20, such as 1 to 15, or 1 to 10, or 1 to 5. In particular aspects, each heteroaliphatic group is —(CH₂)₂OH. In particular aspects, the carboxylic acid group is —CH₂COOH. In a non-limiting exemplary aspect, the amine buffer is bicine.

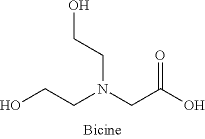 [SMILES: O=C(O)CN(CCO)CCO]

In particular aspects, amine buffers according to the present disclosure do not include, or are other than, carbonate buffers, bicarbonate buffers, phosphate buffers, and/or citric acid/citrate buffers. In some aspects the amine buffer is not, or is other than, 2-amino-2-(hydroxymethyl)propane-1,3-diol, also referred to as tris(hydroxymethyl)aminomethane; triethylammonium bicarbonate; sodium acetate; 4-(2-hydroxyethyl)-1-piperazineethanesulfonic acid (HEPES); and/or borate. Examples included herein that use such buffers are used solely as comparative buffers and are not intended to be included within the scope of amine buffers according to aspects of the present disclosure.

In certain aspects, the amine buffer can have a pH ranging from 7.5 to 9.5, such as 7.5 to 9.4, or 8.0 to 9.5, or 7.5 to 9.4, or 9.0 to 9.5, or 9.0 to 9.4.

In aspects disclosed herein, the amine buffer is present at a concentration ranging from 25 mM to 125 mM, such as 75 mM to 125 mM, or 100 mM to 125 mM relative to the composition's total volume.

### B. Size Exclusion Support

Matrixes according to the present disclosure may comprise a porous size exclusion support. A porous size exclusion support may comprise spherical beads made of a gel or a gel-like material having pores. The pore size range of a porous size exclusion support determines the size of a molecule that may be included or excluded from entering the size exclusion support. Without being bound by a single theory, it currently is believed that when a sample solution is passed through a size exclusion support, at least one small molecule in the sample enters the pores of the size exclusion support and is forced to follow a circuitous path before later exiting the size exclusion support. On the other hand, larger molecules take a relatively direct path through the size exclusion support. Therefore, without being bound by a particular theory of operation, it is currently believed that the difference in flow rates between the small molecules and the large molecules allows for the separation of the faster-flowing large molecules from the slower-flowing small molecules as a sample travels through the size exclusion support.

Some exemplary size exclusion supports are made of agarose, polyacrylamide, cellulosic materials, and/or derivatives thereof. In some aspects, a porous size exclusion support may comprise an agarose support, a polyacrylamide support, a cellulosic material support, or derivatives thereof. In some example aspects, a porous size exclusion support comprises HEC.

In some aspects, the porous size exclusion support is produced by using sufficient HEC to produce a resin with a MWCO greater than or equal to 40 kDa.

In particular disclosed aspects, the porous size exclusion support is produced from a range of 50 grams (g) to 250 grams (g) HEC, such as, but not limited to 50 g HEC to 150 HEC g. In some aspects, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g, 112 g, 113 g, 114 g, 115 g, 116 g, 117 g, 118 g, 119 g, 120 g, 121 g, 122 g, 123 g, 124 g, 125 g, 126 g, 127 g, 128 g, 129 g, 130 g, 131 g, 132 g, 133 g, 134 g, 135 g, 136 g, 137 g, 138 g, 139 g, 140 g, 141 g, 142 g, 143 g, 144 g, 145 g, 146 g, 147 g, 148 g, 149 g 150 g 160 g, 170 g, 180 g, 190 g, 200 g, 201 g, 202 g, 203 g, 204 g, 205 g, 206 g, 207 g, 208 g, 209 g, 210 g, 211 g, 212 g, 213 g, 214 g, 215 g, 216 g, 217 g, 218 g, 219 g, 220 g, 221 g, 222 g, 223 g, 224 g, 225 g, 226 g, 227 g, 228 g, 229 g, 230 g, 240 g, 250 g of HEC can be used to produce the porous size exclusion support. In one non-limiting exemplary aspect 80 g of HEC was used to produce the porous size exclusion support. In another exemplary aspect, 90 g of HEC was used to produce the porous size exclusion support. In another non-limiting exemplary aspect, 108 g of HEC was used to produce the porous size exclusion support. In another non-limiting exemplary aspect, 129 g of HEC was used to produce the porous size exclusion support. In another non-limiting exemplary aspect, 147 g of HEC was used to produce the porous size exclusion support. In another non-limiting exemplary aspect, 216 g of HEC was used to produce the porous size exclusion support.

In some aspects of the disclosure, the porous size exclusion support is produced with 60 g HEC to 130 g HEC at a reaction scale of from greater than 0 liters (L) to 10 liters (L), such as, but not limited to, from greater than 0 L to 5 L, to produce the size exclusion support. In some aspects, 0.5 L to 2 L of resin bed comprising the porous size exclusion support is produced from 60 g HEC to 130 g HEC. In particular disclosed aspect of the present disclosure, 0.5 L to 1.5 L of resin bed comprising the porous size exclusion support is produced from 80 g HEC to 130 g HEC used in a 5 L reaction scale.

In some aspects of the disclosure, the size exclusion support can be crosslinked with a crosslinker such as, but not limited to, an epoxide-containing compound comprising at least one epoxide functional group, such as 1, 2, 3, or 4 epoxide groups. In some aspects, the epoxide-containing compound may comprise one or more halo functional groups, aliphatic functional groups, heteroaliphatic functional groups, or a combination thereof. In some aspects, the heteroaliphatic functional group can comprise a polyethylene glycol (or “PEG”) spacer arm. The epoxide-containing compound can have Formula II,

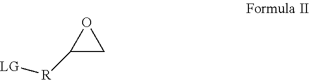

where R is aliphatic (e.g., C₁-C₁₀ alkyl); and LG can be a halo functional group or a glycidol moiety. In one preferred aspect, the epoxide-containing compound can be epichlorohydrin (Epi), having a structure of

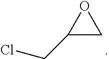

In another preferred aspect, the epoxide-containing compound can be 1,4-butanediglycidylether.

In some aspects, the porous size exclusion support can be crosslinked with a range of 250 milliliters (mL) to 700 milliliters (mL) of the crosslinker. For example, 250 mL, 251 mL, 252 mL, 253 mL, 254 mL, 255 mL, 256 mL, 257 mL, 258 mL, 259 mL, 260 mL, 261 mL, 262 mL, 263 mL, 264 mL, 265 mL, 266 mL, 267 mL, 268 mL, 269 mL, 270 mL, 271 mL, 272 mL, 273 mL, 274 mL, 275 mL, 276 mL, 277 mL, 278 mL, 279 mL, 280 mL, 281 mL, 282 mL, 283 mL, 284 mL, 285 mL, 286 mL, 287 mL, 288 mL, 289 mL, 290 mL, 291 mL, 292 mL, 293 mL, 294 mL, 295 mL, 296 mL, 297 mL, 928 mL, 299 mL, 300 mL, 301 mL, 302 mL, 303 mL, 304 mL, 305 mL, 306 mL, 307 mL, 308 mL, 309 mL, 310 mL, 311 mL, 312 mL, 313 mL, 314 mL, 315 mL, 316 mL, 317 mL, 318 mL, 319 mL, 320 mL, 321 mL, 322 mL, 323 mL, 324 mL, 325 mL, 326 mL, 327 mL, 328 mL, 329 mL, 330 mL, 331 mL, 332 mL, 333 mL, 334 mL, 335 mL, 336 mL, 337 mL, 338 mL, 339 mL, 340 mL, 341 mL, 342 mL, 343 mL, 344 mL, 345 mL, 346 mL, 347 mL, 348 mL, 349 mL, 355 mL, 356 mL, 357 mL, 358 mL, 359 mL, 360 mL, 361 mL, 362 mL, 363 mL, 364 mL, 365 mL, 366 mL, 367 mL, 368 mL, 369 mL, 370 mL, 371 mL, 372 mL, 373 mL, 374 mL, 375 mL, 376 mL, 377 mL, 378 mL, 379 mL, 380 mL, 381 mL, 382 mL, 383 mL, 384 mL, 385 mL, 386 mL, 387 mL, 388 mL, 389 mL, 390 mL, 391 mL, 392 mL, 393 mL, 394 mL, 395 mL, 396 mL, 397 mL, 398 mL, 399 mL, 400 mL, 401 mL, 402 mL, 403 mL, 404 mL, 405 mL, 406 mL, 407 mL, 408 mL, 409 mL, 410 mL, 411 mL, 412 mL, 413 mL, 414 mL, 415 mL, 416 mL, 417 mL, 418 mL, 419 mL, 420 mL, 421 mL, 422 mL, 423 mL, 424 mL, 425 mL, 426 mL, 427 mL, 428 mL, 429 mL, 430 mL, 440 mL, 450 mL, 460 mL, 470 mL, 480 mL, 500 mL, 520 mL, 540 mL, 560 mL, 580 mL, 600 mL, 620 mL, 640 mL, 660 mL, 680 mL, or 700 mL of the crosslinker can be used, with particular aspects using Epi as the crosslinker in the above-stated ranges/amounts.

In one non-limiting exemplary aspect, 265 mL of Epi was used to crosslink the porous size exclusion support. In another non-limiting exemplary aspect, 303 mL of Epi was used to crosslink the porous size exclusion support. In another non-limiting exemplary aspect, 375 mL of Epi was used to crosslink the porous size exclusion support. In another non-limiting exemplary aspect, 397 mL of Epi was used to crosslink the porous size exclusion support. In another non-limiting exemplary aspect, 410 mL of Epi was used to crosslink the porous size exclusion support. In another non-limiting exemplary aspect, 441 mL of Epi was used to crosslink the porous size exclusion support. In yet another non-limiting exemplary aspect, 662 mL of Epi was used to crosslink the porous size exclusion support.

In some aspects, the porous size exclusion support can be crosslinked by providing 250 mL Epi to 700 mL Epi at a reaction scale from greater than 0 L to 5 L. In some particular aspects of the present disclosure, greater than 0 L to 2 L of resin bed comprising the porous size exclusion support can be produced from 250 mL Epi to 400 mL Epi 5 L reaction scale.

In some aspects, the porous size exclusion support has a MWCO greater than or equal to 40 kDa and hence molecules that are excluded have a molecular weight of greater than or equal to 40 kDa. The pore size of the porous size exclusion support may have a MWCO size for excluding from the pores molecules of greater than or equal to 40 kDa to 150 kDa, more typically from 50 kDa to 150 kDa, and even more typically from greater than or equal to 40 kDa to 60 kDa, including a MWCO in between, such as but not limited to, greater than or equal to 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa, 60 kDa, 61 kDa, 62 kDa, 63 kDa, 64 kDa, 65 kDa, 66 kDa, 67 kDa, 68 kDa, 69 kDa, 70 kDa, 71 kDa, 72 kDa, 73 kDa, 74 kDa, 75 kDa, 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa. In one non-limiting exemplary aspect, the porous size exclusion support has a MWCO of greater than or equal to 40 kDa. In another non-limiting exemplary aspect, the porous size exclusion support has a MWCO of 45 kDa. In another non-limiting exemplary aspect, the porous size exclusion support has a MWCO of 50 kDa. In another non-limiting exemplary aspect, the porous size exclusion support has a MWCO of 80 kDa. In another non-limiting exemplary aspect, the porous size exclusion support has a MWCO of 90 kDa.

### C. Cationic Moiety

Matrixes according to the present disclosure may comprise a porous size exclusion support and a cationic moiety. In some aspects of the present disclosure, small molecules, such as, but not limited to, negatively charged molecules can be separated from large molecules, such as, but not limited to, positively charged molecules based on one or more matrix properties. In some aspects, the matrix properties can be the charge and/or the size exclusion properties of the matrix.

In some aspects, the cationic moieties of a matrix of the disclosure are amines, imines, or a combination thereof. In some aspects of the present disclosure, such cationic moieties may comprise an amine-containing polymer; alkylamines, particularly lower alkyl amines (e.g., pentylamines); amine-containing heterocyclic compounds; amine-containing aromatic compounds; or other amine/diamine compounds.

In a non-limiting exemplary aspect, prior to association with the porous size exclusion support, the amine-containing polymer is branched polyethylene imine (PEI), having a base structure as shown below, which when exposed to suitable conditions, such as but not limited to, an amine buffer, becomes charged to provide the cationic moiety.

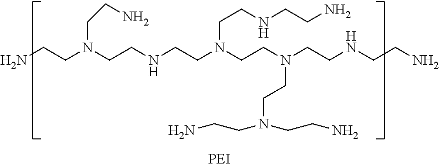

In another non-limiting exemplary aspect, prior to association with the porous size exclusion support, the amine-containing polymer is a diamine comprising one or more polyethylene glycol groups, such as 5,8-dimethyl-4,7,10-trioxatridecane-2,12-diamine (also known commercially as Jeffamine) having a base structure as shown below. When exposed to suitable conditions, such as but not limited to, an amine buffer, the diamine becomes charged to provide the cationic moiety.

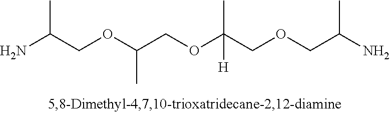 [SMILES: CC(N)COCC(C)OCC(C)OCC(C)N]

In another non-limiting exemplary aspect, prior to association with the porous size exclusion support, the alkyl diamine is diaminopentane (PDA), having a structure NH₂(CH₂)₅NH₂ (and also shown below), which when exposed to suitable conditions, such as but not limited to, an amine buffer, becomes charged to provide the cationic moiety.

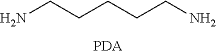

In another non-limiting exemplary aspect, prior to association with the porous size exclusion support, the diamine is N,N diethylethylenediamine (DEED), having a structure as shown below, which when exposed to suitable conditions, such as but not limited to, an amine buffer, becomes charged to provide the cationic moiety.

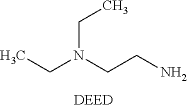

In another non-limiting aspect of the present disclosure, the amine-containing aromatic compound may comprise at least one aromatic ring having from C₃ to C₁₅ and at least one amine-containing compound. Particular disclosed aspects of the present disclosure, the amine-containing aromatic compound can have general Formula III, or an enantiomer, a diastereomer, a tautomer, a salt, a solvate and/or an isotopically substituted derivative thereof

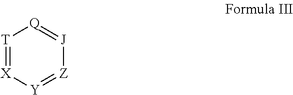

wherein, with reference to Formula III, J, Q, T, X, Y, and Z are the same or different, and each of J, Q, T, X, Y, and Z independently is selected from nitrogen or CR^{c}, wherein R^{c}, for each occurrence, independently is selected from hydrogen, halo, aliphatic, heteroaliphatic, or amino. In some aspects, each R^{c} can be the same or different. In some aspects, prior to association with the porous size exclusion support, each of J, Q, T, X, Y, and Z are CR^{c} and at least two R^{c} groups comprise an amino group and the remaining R^{c} groups are hydrogen. In particular aspects, a compound of Formula III can be selected from, but not limited to, 1,2-diaminobenzene having a structure shown below, 1,3-diaminobenzene having a structure shown below, and/or 1,4-diaminobenzene having a structure shown below. When exposed to suitable conditions, such as but not limited to, an amine buffer, each of these compounds becomes charged to provide the cationic moiety.

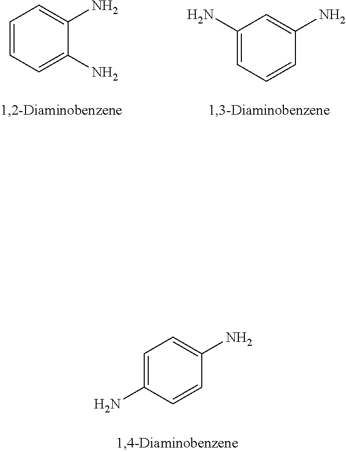 [SMILES: Nc1ccccc1N ; Nc1cccc(N)c1 ; Nc1ccc(N)cc1]

In particular disclosed aspects, an amine-containing heterocyclic compound can be associated with the size exclusion support comprising HEC. In one example, an epoxy amine compound can be associated with the size exclusion support comprising HEC. In some aspects, the amine-containing can be an epoxy amine, such as, but not limited to, (S)-N-boc-2,3-epoxypropylamine having a base structure below, which when exposed to suitable conditions, such as but not limited to, an amine buffer according to the present disclosure, becomes charged to provide the cationic moiety.

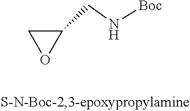 [SMILES: CC(C)(C)OC(=O)NC[C@H]1CO1]

In some aspects, a matrix of the disclosure comprises one or more cationic moieties that are associated with a porous size exclusion support. In some aspects, either a porous size exclusion support or a cationic moiety can include a reactive functional group. For example, a functional group on a porous size exclusion support can be used to interact with and associate with one or more cationic moieties to form a matrix.

In some aspects, a matrix comprises one or more cationic moieties that are immobilized onto at least one size exclusion support. In some aspects, the cationic moiety may be “immobilized” by being covalently bound to the size exclusion support by formation of a covalent bond. In some aspects, the covalent bond can be formed using alkylation or by forming an amide or amine bond between a porous size exclusion support and a cationic moiety. In a non-limiting exemplary aspect, immobilization according to the disclosure is achieved by first oxidizing hydroxyl groups of HEC to aldehyde groups using any suitable oxidizing agent, with some examples using a periodate (IO⁴⁻ or IO₆⁵⁻). The aldehyde groups generated by oxidation can react with terminal amines on a cationic moiety to covalently bind the cationic moiety to the HEC support, and the resulting intermediate may be reduced by using a reducing agent, such as, but not limited to, sodium cyanoborohydride or picoline borane, to form an amine.

In some aspects, the epoxy amine can be immobilized onto the size exclusion support. The epoxy amine can associate with and/or immobilize onto the size exclusion support comprising HEC by reacting with a hydroxyl group provided by the HEC. In some aspects of the disclosure, the epoxy amine compound is a protected amine compound, wherein the protected amine compound is deprotected after associating with or immobilizing onto the size exclusion support.

In some aspects, the cationic moiety associates with the one or more small molecules, such as, but not limited to, a negatively charged small molecule. For example, the negatively charged small molecule can associate with the cationic moiety through interactions and/or bonds, such as, but not limited to, an ionic interaction, a hydrophilic interaction, a hydrophobic interaction, an affinity interaction, hydrogen bonding, Van der Waals forces, and/or covalent bonding.

In particular disclosed aspects, a functional group on a cationic moiety can react with a functional group on a porous size exclusion support and/or a small molecule, such as but not limited to, a negatively charged small molecule, to be separated or extracted from the at least one large molecule. Functional groups can include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, and/or any other functional group suitable for associating and/or interacting cationic moieties with the porous size exclusion support and/or small molecule.

In another aspect, functional groups include at least one reactive group represented by either Rₓ, which represents a reactive functional moiety; or (-L-Rₓ), which represents a reactive functional moiety Rₓ that is attached to either a porous size exclusion support or a moiety by a covalent linkage “L.” The reactive group functions as the site of association, attachment and/or interaction with a moiety or a small molecule wherein the reactive group chemically reacts with an appropriate reactive or functional group on the porous size exclusion support, the moiety, or the small molecule. In a non-limiting exemplary aspect, a reactive group or a functional group can be an acrylamide, an activated ester of a carboxylic acid, an acyl halide group, an acyl azide, an acyl nitrile, an aldehyde, an alkyl halide, an anhydride, an aniline, an aryl halide, an azide, an aziridine, a boronate, a thioboronate group, a carboxylic acid, a diazoalkane, a haloacetamide, a halotriazine, a hydrazine, a hydrazide, an imido ester, an isocyanate, an isothiocyanate, a maleimide, a phosphoramidite, a sulfonyl halide, a thiol group, a sulfide group, a disulfide group, an epoxide group, and episulfide group, a thioester group, an alcohol group, an activated alcohol group, a phosphate group, a phosphate ester group, and/or a photoactivatable group.

In another non-limiting exemplary aspect, a reactive group or functional group can comprise electrophiles and/or nucleophiles and can, in some aspects, generate a covalent linkage between them. Exemplary electrophiles and nucleophile functional groups can an aryloxy group or aryloxy substituted one or more times by electron-withdrawing substituents such as nitro, fluoro, chloro, cyano, trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, an acyl halide group, an acyl nitrile, an aldehyde, an alkyl halide, an anhydride, an aryl halide, an aziridine, a diazoalkane, a haloacetamide, a halotriazine, an isocyanate, an isothiocyanate, a maleimide, a phosphoramidite, a sulfonyl halide, a sulfide group, a disulfide group, an epoxide group, and episulfide group, a thioester group, an activated alcohol group, a phosphate group, a phosphate ester group, and a photoactivatable group. Acyl azides can also rearrange to isocyanates.

In some aspects, the reactive group further comprises a linker, L, in addition to the reactive functional moiety. The linker can be used to covalently attach a reactive functional group. When present, the linker is a single covalent bond or a series of stable bonds. A reactive functional moiety may be directly attached (where the linker is a single bond) through a series of stable bonds, to the solid support, the moiety or to the small molecule. When the linker is a series of stable covalent bonds the linker typically incorporates several nonhydrogen atoms selected from C, N, O, S, Si, B and P. In addition, the covalent linkage can incorporate a platinum atom, such as described in U.S. Pat. No. 5,714,327. When the linker is not a single covalent bond, the linker may be any combination of stable chemical bonds, optionally including, single, double, triple, or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In a non-limiting exemplary aspect, the linker incorporates less than 15 nonhydrogen atoms and is composed of a combination of ether, thioether, thiourea, amine, ester, carboxamide, sulfonamide, hydrazide, aromatic, and/or heteroaromatic bonds. Typically, the linker is a single covalent bond or a combination of single carbon-carbon bonds and carboxamide, sulfonamide, or thioether bonds. In some aspects, the following moieties can be found in the linker: ether, thioether, carboxamide, thiourea, sulfonamide, urea, urethane, hydrazine, alkyl, aryl, heteroaryl, alkoxy, cycloalkyl and amine moieties. Examples of L include substituted or unsubstituted polymethylene, arylene, alkylarylene, aryl, or arylthiol.

Any combination of linkers can be used to attach functional or reactive groups. Where the reactive group is a maleimide or haloacetamide, the resulting compound is particularly useful for conjugation to thiol-containing substances. Where the reactive group is a hydrazide, the resulting compound is particularly useful for conjugation to periodate-oxidized carbohydrates and glycoproteins.

In some aspects, the cationic moiety may comprise an open and/or unreacted amine. For example, a matrix comprising a size exclusion support and a cationic moiety, such as, but not limited to, PDA, may comprise an open/unreacted amine. In particular disclosed aspects, the open/unreacted amine on PDA can be reacted with a polysaccharide derived molecule having a molecular weight range from 0 to 2,000,000. In one non-limiting exemplary aspect, the open and/or unreacted amine on the cationic moiety PDA is reacted with dextran having a molecular weight of 1,000,000.

In some aspects, the concentration of the cationic moiety that is added to the matrix can be in the range of from 50 mg/mL to 175 mg/mL. In some aspects the concentration of the cationic moiety is from 50 mg/mL to 100 mg/mL, from 50 mg/mL to 75 mg/mL, from 50 mg/mL to 150 mg/mL, from 100 mg/mL to 155 mg/mL, from 130 mg/mL to 175 mg/mL. In one non-limiting exemplary aspect, the concentration of the cationic moiety can be 50 mg/mL. In another non-limiting exemplary aspect, the concentration of the cationic moiety can be 75 mg/mL. In another non-limiting exemplary aspect, the concentration of the cationic moiety can be 150 mg/mL.

In certain aspects, the porous size exclusion support is produced with 129 grams of HEC and 265 milliliters of Epi to make 1.5 liters of resin bed, and wherein the HEC is modified with a plurality of cationic PDA moieties to form the matrix having an overall positive charge.

In some aspects, the porous size exclusion support is produced with 129 grams of HEC and 265 milliliters of Epi to make 1.5 liters of resin bed, and wherein the HEC is modified with a plurality of cationic PEI moieties to form the matrix having an overall positive charge.

In particular aspects, the porous size exclusion support is produced with 129 grams of HEC and 265 milliliters of Epi to make 1.5 liters of resin bed, and wherein the HEC is modified with a plurality of DEED moieties to form the matrix having an overall positive charge.

### VI. Kit

The present disclosure also describes kits for implementing the methods disclosed herein and/or kits that contain compositions disclosed herein. In some aspects, the present disclosure describes a kit for isolating conjugatable large molecules. In certain aspects, components of the kit are used to separate large molecules from small molecules in a sample using differences in one or more properties and equilibrates the large molecule to produce conjugatable large molecules. In certain aspects, components of the kit can separate a large molecule from a small molecule using differences in properties such as, but not limited to, the size of the molecules, charge of the molecules, the isoelectric point of the molecules, and/or any combination of these properties.

In certain aspects, the kit may comprise a first container comprising (i) a porous size exclusion support, and (ii) one or more cationic moieties associated with the porous size exclusion support, wherein the matrix can separate molecules in a sample by using a molecular weight of the molecules, a charge of the molecules, an isoelectric point (pI) of the molecules, or a combination thereof; and a second container comprising an amine buffer according to the present disclosure.

In some aspects, the kit can further comprise a dye such as, but not limited to, a fluorescent dye. In certain aspects, the dye can be packaged in a third container, or can be included in the first or second container.

In certain aspects, the kit can further comprise one or more receptacles for recovering the isolated large molecules. In some aspects, the receptacle is adaptable or can be configured to receive fluid flowing from the first or second container. In particular aspects, the receptacle is removably coupled to the first and/or second container and can be detached from the first for second container so that a user can collect the flow-through.

In aspects disclosed herein, the one or more containers in a kit can be a columnar container, a tube, a multi-well tube, a multi-well plate or a multi-well filter plate. Exemplary containers include, but are not limited to, a vial, flask, bottle, syringe, a test tube, a spin column, a multi-well plate, a multi-well filter plate, a micro-well plate, or a micro-well filter plate. Where there is more than one container in a kit, they may be packaged together if suitable or the kit will generally contain a second, third or other additional container into which the additional components may be separately placed. In some aspects, certain combinations of components may be packaged together in one container. A kit can also include a component for containing any reagent containers in close confinement for commercial sale. Such containers may include injection or blow-molded plastic containers into which the desired vials are retained.

In some aspects, the kit can be pre-filled with one or more components to process a sample and may be suitably aliquoted into appropriate chambers. A kit or containers thereof may have a seal to keep the internal compartments and any contents therein sterile and spill proof.

Some components of a kit are provided in one and/or more liquid solutions. Liquid solution may be non-aqueous solution, an aqueous solution, and may be a sterile solution. Components of the kit may also be provided as dried powder(s). When reagents and/or components are provided as a dry powder, the powder can be reconstituted by the addition of a suitable solvent. It is envisioned that a suitable solvent may also be provided in another container means.

A kit of the disclosure may also include instructions for employing the kit components and may also have instructions for the use of any other reagent not included in the kit. Instructions can include variations that can be implemented.

### VII. Overview of Several Aspects

Disclosed herein are aspects of a method, comprising combining an amine buffer according to Formula I with a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof, wherein Formula I is

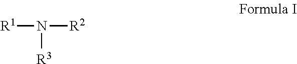

wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof.

In some aspects of the present disclosure, the amine buffer is a secondary amine or a tertiary amine.

In any or all of the above aspects, the amine buffer is a secondary amine and at least one of R¹, R², and R³ is a heteroaliphatic group.

In any or all of the above aspects, the heteroaliphatic group is monohydric, dihydric, trihydric, or polyhydric.

In any or all of the above aspects, the amine buffer is tricine.

In any or all of the above aspects, the amine buffer is a tertiary amine and two of R¹, R², and R³ independently are a heteroaliphatic group.

In any or all of the above aspects, the heteroaliphatic group is monohydric, dihydric, trihydric, or polyhydric.

In any or all of the above aspects, the amine buffer is bicine.

In any or all of the above aspects, the amine buffer has a pH ranging from 7.0 to 9.5.

In any or all of the above aspects, the method further comprises adding a dye.

In any or all of the above aspects, the dye is a fluorescent dye.

Also disclosed herein is a method, comprising: exposing a sample to a matrix and a buffer, wherein (a) the matrix comprises (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof; and (b) the buffer is the amine buffer disclosed herein.

In some aspects of the present disclosure, the sample is exposed to a composition comprising the matrix and the buffer.

In any or all of the above aspects, the (i) the sample is first exposed to the matrix and then exposed to the buffer; or (ii) the sample is first exposed to the buffer and then exposed to the matrix.

In any or all of the above aspects, the sample comprises an antibody.

In any or all of the above aspects, the method further comprises isolating the antibody.

In any or all of the above aspects, the method further comprises adding a dye to facilitate forming a conjugate with the antibody.

A kit is also disclosed herein, the kit comprising a first container comprising a matrix comprising (i) a porous size exclusion support, and (ii) one or more cationic moieties associated with the porous size exclusion support, wherein the matrix can separate one or more molecules in a sample by using a molecular weight of the one or more molecules, a charge of the one or more molecules, an isoelectric point (pI) of the one or more molecules, or a combination thereof; and a second container comprising the amine buffer disclosed herein.

In some aspects of the present disclosure, the kit further comprises a dye.

A composition is also disclosed herein, the composition comprising an amine buffer according to Formula I,

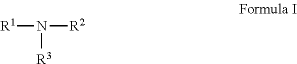

wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof; and a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof.

In some aspects of the present disclosure, the composition further comprises an antibody, dye, or both.

In any or all of the above aspects, the porous size exclusion support has a molecular weight cut-off of greater than or equal to 40 kDa.

In any or all of the above aspects, the amine buffer is a secondary amine or a tertiary amine.

In any or all of the above aspects, the amine buffer is a secondary amine and at least one of R¹, R², and R³ is a heteroaliphatic group.

In any or all of the above aspects, the heteroaliphatic group is monohydric, dihydric, trihydric, or polyhydric.

In any or all of the above aspects, the amine buffer is tricine.

In any or all of the above aspects, the amine buffer is a tertiary amine and two of R¹, R², and R³ independently are a heteroaliphatic group.

In any or all of the above aspects, the heteroaliphatic group is monohydric, dihydric, trihydric, or polyhydric.

In any or all of the above aspects, the amine buffer is bicine.

In any or all of the above aspects, wherein the amine buffer is present at a concentration ranging from 25 mM to 125 mM relative to the composition's total volume.

In any or all of the above aspects, the one or more cationic moieties are covalently bound to the porous size exclusion support.

In any or all of the above aspects, a plurality of cationic moieties are covalently bound to the porous size exclusion support.

In any or all of the above aspects, the one or more cationic moieties comprise an amine, a diamine, a polyamine, amine-containing heterocyclic compound, amine-containing aromatic compound, or an amine-containing polymer.

In any or all of the above aspects, the one or more cationic moieties are polyethylene imine, diaminopentane, 5,8-dimethyl-4,7,10-trioxatridecane-2,12-diamine, 1,2-diaminobenzene, 1,3-diaminobenzene, 1,4-diaminobenzene, or N,N-diethylethylenediamine.

In any or all of the above aspects, the porous size exclusion support comprises hydroxyethyl cellulose.

In any or all of the above aspects, the porous size exclusion support is crosslinked with epichlorohydrin.

Also disclosed herein is a composition comprising a matrix (i) a porous size exclusion support, and (ii) one or more cationic moieties associated with the porous size exclusion support, wherein the matrix can separate one or more molecules in a sample by using a molecular weight of the one or more molecules, a charge of the one or more molecules, an isoelectric point (pI) of the one or more molecules, or a combination thereof; an antibody; and a means for equilibrating the antibody for conjugation, wherein the means does not comprise 2-amino-2-(hydroxymethyl)propane-1,3-diol, triethylammonium bicarbonate, sodium acetate, or 4-(2-hydroxyethyl)-1-piperazineethanesulfonic acid.

The foregoing and other objects, features, and advantages of the present disclosure will become more apparent from the following detailed description, which proceeds with reference to the accompanying figures.

### VIII. Examples

Aspects of the present teachings can be further understood in light of the following examples.

Matrixes comprising at least one size exclusion support and one or more cationic moieties for separating and/or extracting one or more small molecules from a sample that can associate with the one or more small molecules using differences in one or more properties, such as but not limited to, the size of the molecules, the charge of the molecules, the isoelectric point of the molecules, and/or any combination of these properties were prepared and tested.

In some aspects, exemplary size exclusion supports were modified with cationic moieties comprising functional groups that can associate with negatively charged small molecules by either ionic interaction, hydrophilic interaction, or any other interaction(s) that can associate with and thereby remove negatively charged small molecules from a sample while allowing at least one large positively charged molecules in the sample to be excluded and collected.

In the following examples, 642 μL of resin bed were spun at 3000×G for 1 minute to remove storage solution. Resin was equilibrated with buffer (3×300 μL). The resin was then spun at 3000×G for 1 minute to remove buffer for the first two spins. For the third and final equilibration, the resin was spun at 3000×G for 2 minutes.

100 μL of BSA (antibody mixture) was added to the resin and spun at 3000×G, 2 minutes to collect the flowthrough (“FT”). In certain evaluations, before conjugating, NHS activated dyes to the antibody, the resins were spiked with 1 M Borate, pH 9.2 at 10% volume of the FT. In some evaluations, the NHS dye is added directly to the collected antibody without spiking the resin. The NHS dyes were added at molar excesses to the antibody per optimized protocol for each respective dye and reacted with the dye with the antibody for 30 minutes at room temperature. Excess dye was removed using Zeba dye and biotin removal columns. For this, buffer exchange 562 μL of Zeba dye and biotin removal columns using phosphate-buffered saline (PBS) (3×) using 300 μL of the buffer. Spin speeds were 1000×G and spin times were 1 minute for the first two equilibrations and 2 minutes for the final equilibration.

The conjugates were prepared by diluting them and the “start” sample at a 1:10 ratio in the amine buffer. 10 μL was added per well to a 4-20% Tris Glycine gel, which was run at 200 V for 45-50 minutes. The gel was imaged using the iBright imager in fluorescence protein mode, and the bands were analyzed with iBright image analysis software.

To run the conjugates on a gel, prepare 1:10 dilution of the conjugate and the “start” sample in fluorescence compatible sample buffer. Add 10 uL/well to a 4-20% Tris Glycine gel. Run the gel at 200 V for 45 minutes-50 minutes. Image the gel on iBright imager using fluorescence protein mode. Analyze the band using iBright image analysis software.

### Dye-to-Protein (DIP) of Cleaned-Up Conjugates:

A 280 of the cleaned-up conjugate was measured and the “Start” protein (Antibody-BSA mix). A max (dye dependent) was measured of the conjugate and the “Start” protein. Equation 1 was used to calculate the D/P of the cleaned-up conjugate and the antibody BSA mix sample.

$$Protein Concentration ( M ) = \frac{{A}_{280} - ( {A}_{max} \times CF )}{ε} \times Dilution Factor \qquad Equation 1$$

For Equation 1, E is the protein molar extinction coefficient (e.g., the molar coefficient of IgG is ~210,000 M⁻¹ cm⁻¹); Aₘₐₓ is the absorbance (A) of a dye solution measured at the wavelength maximum (λₘₐₓ) for the dye molecule; CF is the correction factor that adjusts for the amount of absorbance at 280 nm caused by the dye (see Table 1); and the dilution factor is the extent (if any) to which the protein dye:sample was diluted for absorbance measurement.

The degree of labeling was determined according to Equation 2,

$$Moles of Dye per Mole Protein = \frac{{A}_{max} of Labeled Protein}{{ε}^{′} \times Protein Concentration ( M )} \times Dilution Factor \qquad Equation 2$$

where ε′ is the molar extinction coefficient of the fluorescent dye.

In the following examples, bicine, tricine, and sodium acetate (comparator) were evaluated under different conditions. Quantitative iBright analysis of SDS-Page results provided an accurate estimation of BSA and antibody recovery rates when the Antibody-BSA mixture was spun through the resin. Moreover, the following examples address the issue of producing isolated conjugatable antibodies without further sample preparation steps. Furthermore, in some of the following examples, BSA removal and IgG purification kits were used. For example, the Pierce™ Antibody Clean-up Kit commercially sold by Thermo Scientific™ was used in some of the following examples.

While these exemplary small molecules and the listed molecular weight ranges were used in the experimental demonstration, aspects of the present disclosure are not limited to either these small molecules or molecular weight ranges and the teachings herein enable those in the art to make and use matrixes and kits for removal of a variety of small molecules and molecular weight ranges.

### Example 1

In this example, non-spiked bicine, bicine spiked with borate, non-spiked sodium acetate, and sodium acetate spiked with borate were used for equilibrating a matrix comprising (i) a porous size exclusion support and (ii) one or more cationic moieties were investigated for removing BSA and producing a conjugatable antibody from sample. Table 1 shows the buffers, PH, concentration, and whether the evaluation was spiked with 1 M borate.

TABLE 1

| Lane | Evaluation | | | Buffer | Spiked with 1M |
|---|---|---|---|---|---|
| No. | No. | Buffer | pH | Concentration | Borate, pH 9.4 |
| 1 | 1 | Bicine | 9.2 | 25 mM | No |
| 2 | 2 | Bicine | 9.2 | 25 mM | Yes |
| 3 | 3 | Bicine | 9.2 | 50 mM | No |
| 4 | 4 | Bicine | 9.2 | 50 mM | Yes |
| 5 | 5 | Bicine | 9.2 | 75 mM | No |
| 6 | 6 | Bicine | 9.2 | 75 mM | Yes |
| 7 | 7 | Sodium | 7 | 75 mM | No |
| | | Acetate | | | |
| 8 | 8 | Sodium | 7 | 75 mM | Yes |
| | | Acetate | | | |

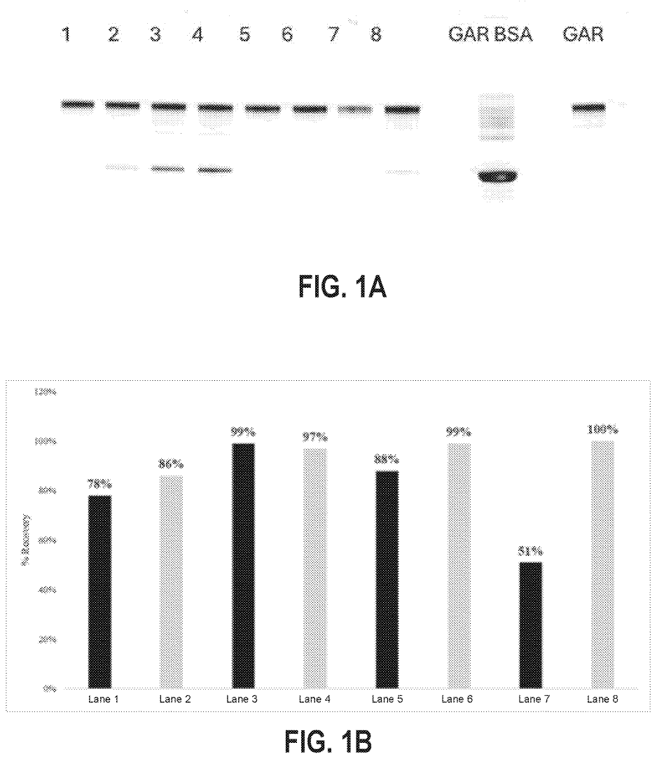

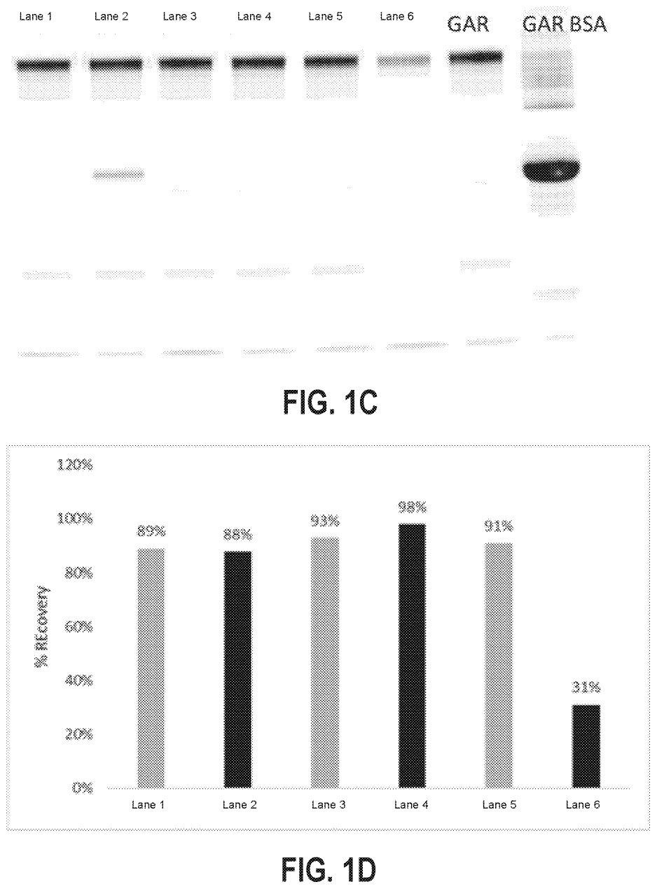

FIG. 1A is a digital image of a fluorescent gel showing the recovery of Goat Anti Rabbit-DyLight® 650 (GAR-Dy 650) conjugate using non-spiked 25 mM bicine (Lane 1); 25 mM bicine spiked with 1 M borate (Lane 2); non-spiked 50 mM bicine (Lane 3); 50 mM bicine spiked with 1 M borate (Lane 4); non-spiked 75 mM bicine (Lane 5); 75 mM bicine spiked with 1 M borate (Lane 6); non-spiked 75 mM spodium acetate (Lane 7); and 75 mM sodium acetate spiked with 1 M borate (Lane 8); the results demonstrate that non-spiked and spiked bicine used at various concentrations produced conjugatable Goat Anti Rabbit antibody (GAR) and subsequent conjugation had a desirable recovery of a GAR-Dy 650 conjugate. FIG. 1B is a bar graph showing the GAR Dy 650 recovery (%) of the fluorescent gel of FIG. 1A and demonstrating a 78% recovery for non-spiked 25 mM bicine (Lane 1); 86% recovery for 25 mM bicine spiked with 1 M borate (Lane 2); 99% recovery for non-spiked 50 mM bicine (Lane 3); 97% recovery for 50 mM bicine spiked with 1 M borate (Lane 4); 88% recovery for non-spiked 75 mM bicine (Lane 5); 99% recovery for 75 mM bicine spiked with 1 M borate (Lane 6); 51% recovery for non-spiked 75 mM sodium acetate (Lane 7); and 100% recovery for 75 mM sodium acetate spiked with 1 M borate (Lane 8).

FIGS. 1A and 1B demonstrate a higher recovery of the Goat Anti Rabbit DyLight® 650 (GAR Dy 650) conjugate when using bicine at concentrations ranging from 25 mM to 75 mM. Furthermore, evaluations using non-spiked and spiked bicine exhibited similar GAR Dy 650 conjugate recovery (%). Evaluations using non-spiked sodium acetate for equilibration, however, demonstrated a 51% recovery; whereas the evaluation using sodium acetate spiked with 1 M borate demonstrated a 100% GAR Dy 650 conjugate recovery. Accordingly, FIGS. 1A and 1B show that bicine concentrations (25-75 mM) achieved high GAR DyLight® 650 conjugate recovery, with similar recovery percentages for both spiked and non-spiked bicine. In contrast, non-spiked sodium acetate resulted in only 51% recovery, while sodium acetate spiked with 1 M borate achieved 100% recovery. These results demonstrate that bicine achieved efficient BSA removal and produced conjugatable antibodies without additional sample preparation between BSA removal and conjugation.

Higher concentrations of bicine and sodium acetate were then investigated as equilibration and conjugation buffers. Table 2 shows the buffers, PH, concentration, and whether the evaluation was spiked with 1 M borate.

TABLE 2

| | | | | | Spiked |
|---|---|---|---|---|---|
| | | | | | with 1M |
| Lane | Evaluation | | | Buffer | Borate, |
| No. | No. | Buffer | pH | Concentration | pH 9.4 |
| 1 | 9 | Bicine | 9.2 | 125 mM | Yes |
| 2 | 10 | Bicine | 9.2 | 125 mM | No |
| 3 | 11 | Bicine | 9.2 | 150 mM | Yes |
| 4 | 12 | Bicine | 9.2 | 150 mM | No |
| 5 | 7 | Sodium Acetate | 7 | 75 mM | No |
| 6 | 8 | Sodium Acetate | 7 | 75 mM | Yes |

FIG. 1C is a digital image of a fluorescent gel showing the recovery GAR-Dy 650 conjugate using 125 mM bicine spiked with 1 M borate (Lane 1); non-spiked 125 mM bicine (Lane 2); 150 mM bicine spiked with 1 M borate (Lane 3); 150 mM bicine spiked with 1 M borate (Lane 4); non-spiked 75 mM sodium acetate (Lane 5); and 75 mM sodium acetate spiked with 1 M borate (Lane 6); the results demonstrate that the non-spiked and spiked bicine buffers at higher concentrations produced conjugatable GAR and subsequent conjugation had a desirable recovery of a GAR-Dy 650 conjugate. FIG. 1D is a bar graph showing the GAR-Dy 650 recovery (%) of the fluorescent gel of FIG. 1C and demonstrating a 89% recovery for 125 mM bicine spiked with 1 M borate (Lane 1); 88% recovery for non-spiked 125 mM bicine (Lane 2); 93% recovery for 150 mM bicine spiked with 1 M borate (Lane 3); 98% recovery for 150 mM bicine spiked with 1 M borate (Lane 4); 31% recovery for non-spiked 75 mM sodium acetate (Lane 5); and 91% recovery for 75 mM sodium acetate spiked with 1 M borate (Lane 6).

FIGS. 1C and 1D show that bicine at concentrations of 125-150 mM achieved high recovery of Goat Anti Rabbit DyLight® 650 (GAR Dy 650) conjugates, with consistent recovery percentages for both spiked and non-spiked bicine. These results demonstrate that higher bicine concentrations removed BSA and isolated conjugatable antibodies, achieving conjugation without requiring additional preparation steps between clean-up and conjugation.

This example demonstrates that bicine at concentrations of 25 mM-150 mM (FIGS. demonstrated desirable BSA removal and high recovery of GAR DyLight® 650 conjugates, with similar results for spiked and non-spiked solutions. Sodium acetate alone achieved only 51% recovery, whereas sodium acetate with 1 M borate reached 100%. As such, bicine produced conjugatable antibodies without additional preparation steps between BSA removal and conjugation.

### Example 2

In this example, bicine (125 mM), tricine (125 mM), and sodium acetate (75 mM) at pH 9.2 were investigated for producing conjugatable antibodies. Table 3 shows the buffers, PH, concentration, and whether the evaluation was spiked with 1 M borate.

TABLE 3

| Lane | Evaluation | | | Buffer | Spiked with 1M |
|---|---|---|---|---|---|
| No. | No. | Buffer | pH | Concentration | Borate, pH 9.4 |
| 1 | 10 | Bicine | 9.2 | 125 mM | No |
| 2 | 13 | Tricine | 9.2 | 125 mM | No |
| 3 | 14 | Sodium | 9.2 | 75 mM | No |
| | | Acetate | | | |

GAR-647 conjugate and GAR-800 conjugate were prepared by using the buffers in Table 3 to equilibrate matrix for BSA removal and the isolated antibodies (GAR) were subsequently conjugated to NHS DyLight® 647 and NHS DyLight® 800 without any spiking.

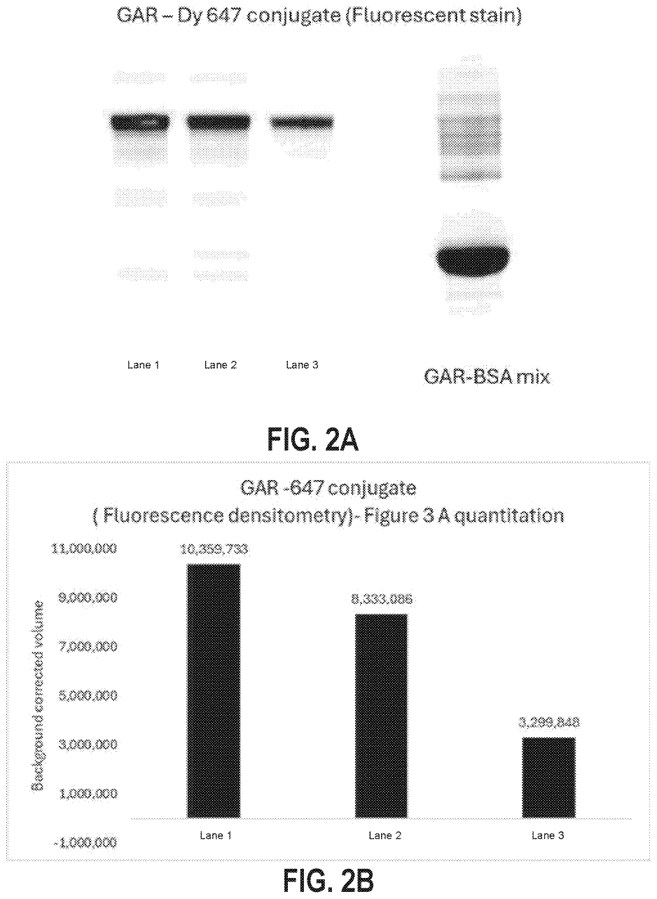

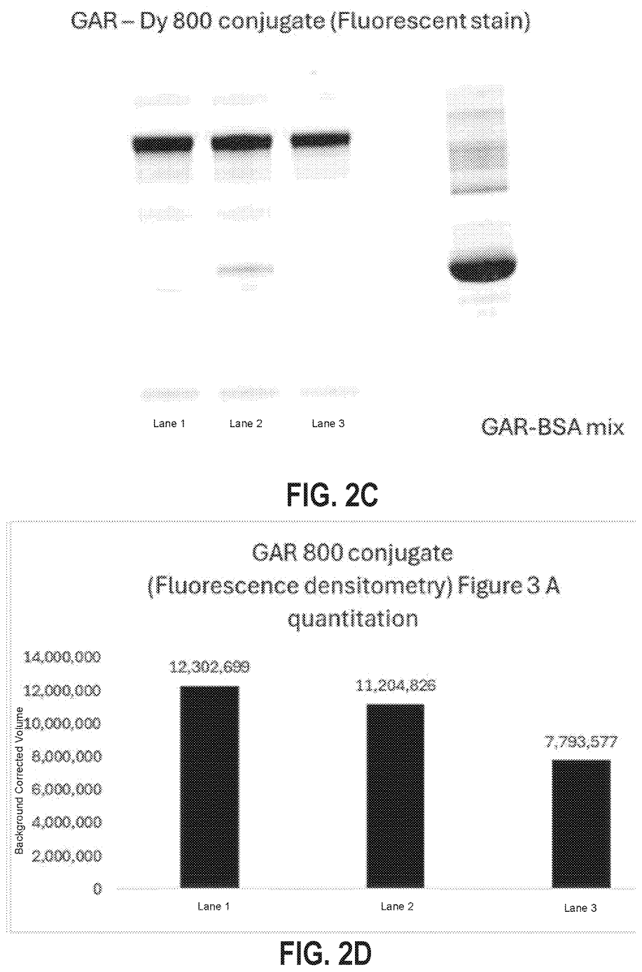

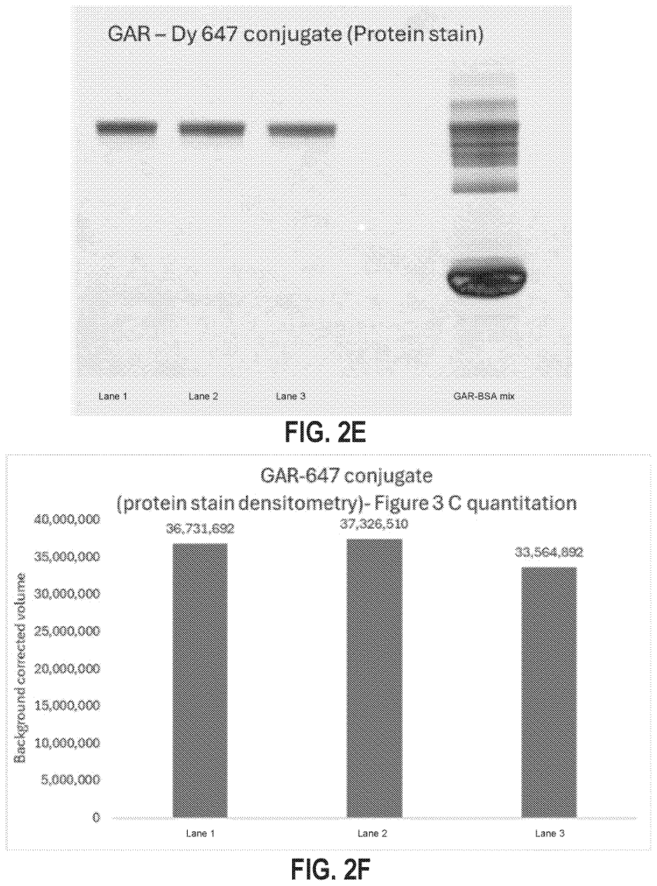

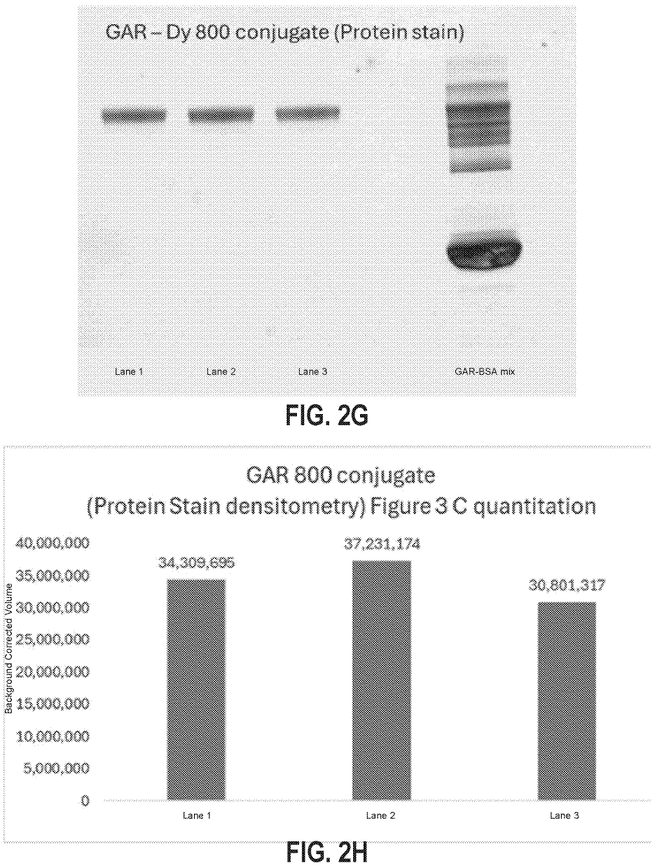

FIG. 2A is a digital image of a fluorescent gel showing the recovery of GAR-Dy 647 conjugate (fluorescent stain) using non-spiked 125 mM bicine (Lane 1); non-spiked 125 mM tricine (Lane 2); and non-spiked 75 mM sodium acetate (Lane 3); the results demonstrate that the lanes using non-spiked bicine and non-spiked tricine produced conjugatable GAR and subsequent conjugation to NHS DyLight® 647 exhibited a higher intensity relative to the lane using sodium acetate. FIG. 2B is a bar graph showing the GAR-647 conjugate quantitation (fluorescence densitometry) results for the gel of FIG. 2A and resulting background corrected volumes for non-spiked 125 mM bicine (Lane 1); non-spiked 125 mM tricine (Lane 2); and non-spiked 75 mM sodium acetate (Lane 3).

FIG. 2C is a digital image of a fluorescent gel showing the recovery of GAR-Dy 800 conjugate (fluorescent stain) using non-spiked 125 mM bicine (Lane 1); non-spiked 125 mM tricine (Lane 2); and non-spiked 75 mM sodium acetate (Lane 3); the results demonstrate that the lanes using bicine and tricine produced conjugatable GAR and subsequent conjugation to NHS DyLight® 800 exhibited a higher intensity relative to the lane using sodium acetate. FIG. 2D is a bar graph showing the GAR-800 conjugate quantitation (fluorescence densitometry) results for the gel of FIG. 2C and resulting background corrected volumes for non-spiked 125 mM bicine (Lane 1); non-spiked 125 mM tricine (Lane 2); and non-spiked 75 mM sodium acetate (Lane 3).

FIGS. 2A-2D demonstrate that non-spiked 125 mM bicine and tricine resulted in higher recovery and conjugation efficiency of GAR-DyLight® 647 compared to 75 mM sodium acetate, as shown by both fluorescent gel imaging and fluorescence densitometry. Furthermore, non-spiked 125 mM bicine and tricine produced superior recovery and conjugation of GAR-DyLight® 800 relative to sodium acetate. Accordingly, bicine and tricine achieved desirable conjugatable antibody recovery and labeling.

FIG. 2E is a digital image of a Coomassie protein-stained gel showing the recovery of GAR-Dy 647 conjugate (protein stain) using non-spiked 125 mM bicine (Lane 1); non-spiked 125 mM tricine (Lane 2); and non-spiked 75 mM sodium acetate (Lane 3); results demonstrate that the evaluations using bicine and tricine produced conjugatable GAR with subsequent conjugation to NHS DyLight® 647 had similar recoveries when stained with Coomasie protein stain and therefore had similar amounts of conjugate present. FIG. 2F is a bar graph showing the GAR-Dy 647 conjugate quantitation (fluorescence densitometry) results for the gel of FIG. 2E and resulting background corrected volumes for non-spiked 125 mM bicine (Lane 1); non-spiked 125 mM tricine (Lane 2); and non-spiked 75 mM sodium acetate (Lane 3).

FIG. 2G is a digital image of a Coomassie protein-stained gel showing the recovery of GAR-Dy 800 conjugate (protein stain) using non-spiked 125 mM bicine (Lane 1); non-spiked 125 mM tricine (Lane 2); and non-spiked 75 mM sodium acetate (Lane 3); results demonstrate that the evaluations using bicine and tricine produced conjugatable GAR with subsequent conjugation to NHS DyLight® 800 had similar recoveries when stained with Coomasie protein stain and therefore had similar amounts of conjugate present. FIG. 2H is a bar graph showing the GAR-Dy 800 conjugate quantitation (fluorescence densitometry) of the gel of FIG. 2G and demonstrating background corrected volume of 34,309,695 for non-spiked 125 mM bicine (Lane 1); 37,231,174 for non-spiked 125 mM tricine (Lane 2); and 30,801,317 for non-spiked 75 mM sodium acetate (Lane 3).

FIGS. 2E-2H show that both non-spiked 125 mM bicine and tricine achieved similar amounts of conjugate recovery, as reflected in the staining and background-corrected volumes. Conversely, 75 mM sodium acetate resulted in lower recovery. Moreover, GAR-Dy 800 conjugates showed comparable recovery and greater recovery rates with bicine and tricine.

As such, both non-spiked bicine and tricine at pH 9 demonstrated higher recovery and conjugation efficiency of GAR-DyLight® 647 and GAR-DyLight® 800 compared to 75 mM sodium acetate. Similarly, non-spiked 125 mM bicine and tricine achieved comparable recovery amounts for GAR-Dy 647 and GAR-Dy 800. In contrast, 75 mM sodium acetate yielded lower recovery, demonstrating that bicine and tricine achieved greater conjugatable antibody recovery and labeling.

### Example 3

In this example, bicine and tricine were investigating for producing conjugatable antibodies. Specifically Rabbit IgG, Mouse IgG, and Mouse IgG1 were isolated using the buffers shown in Table 4 to equilibrate the matrix for removing BSA and subsequently conjugated to three different fluorescent dyes, NHS DyLight® 488, NHS DyLight® 680, and NHS DyLight® 405 without any spiking.

TABLE 4

| Lane | Evaluation | | | Buffer | Spiked with 1M |
|---|---|---|---|---|---|
| No. | No. | Buffer | pH | Concentration | Borate, pH 9.4 |
| 1 | 10 | Bicine | 9.2 | 125 mM | No |
| 2 | 13 | Tricine | 9.2 | 125 mM | No |

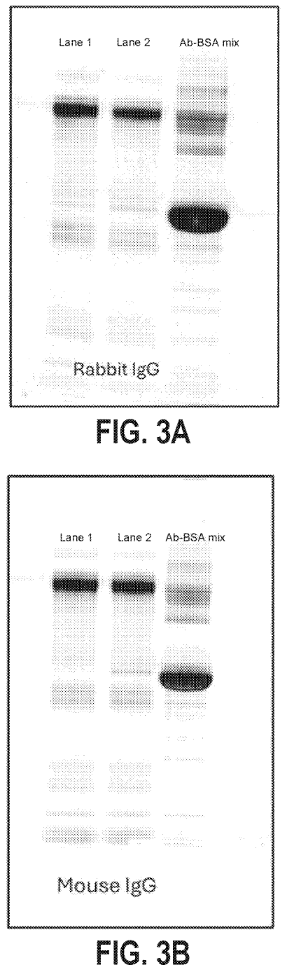

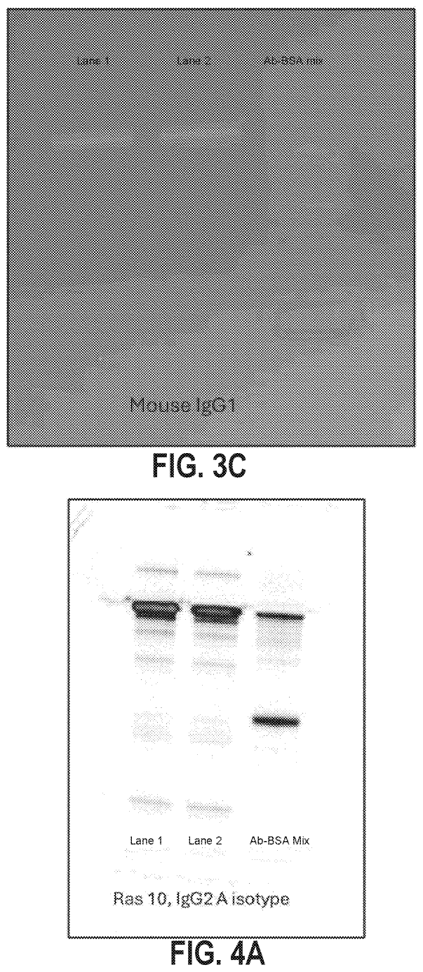

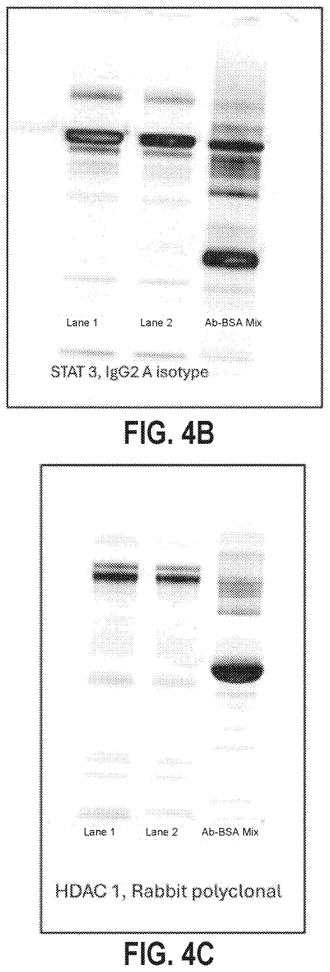

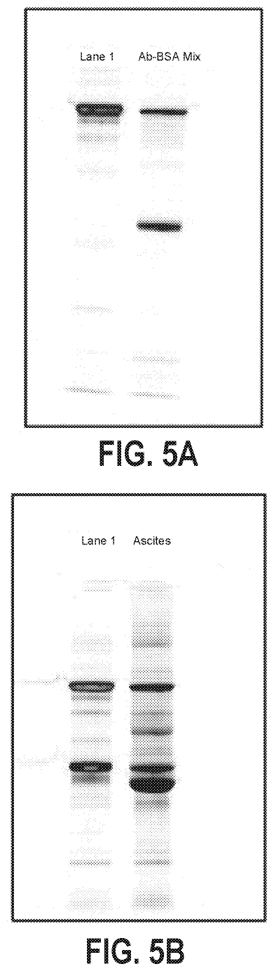

FIG. 3A is a digital image of a fluorescent gel showing the recovery of isolated conjugatable rabbit IgG subsequently conjugated to NHS DyLight® 488 using non-spiked 125 mM bicine (lane 1) and non-spiked 125 mM tricine (Lane 2); results demonstrate that bicine and tricine exhibited a desirable fluorescent signal for the rabbit IgG antibody-NHS DyLight® 488 conjugate.

FIG. 3B is a digital image of a fluorescent gel showing the recovery of isolated conjugatable mouse IgG subsequently conjugated to NHS DyLight® 680 using non-spiked 125 mM bicine (Lane 1) and non-spiked 125 mM tricine (Lane 2); results demonstrate that bicine and tricine exhibited a desirable fluorescent signal for the mouse IgG antibody-NHS DyLight® 680 conjugate.

FIG. 3C is a digital image of a fluorescent gel showing the recovery of isolated conjugatable mouse IgG1 subsequently conjugated to NHS DyLight® 405 using non-spiked 125 mM bicine (Lane 1) and non-spiked 125 mM tricine (Lane 2); results demonstrate that bicine and tricine exhibited a desirable fluorescent signal for the mouse IgG1 antibody-NHS DyLight® 405 conjugate.

FIGS. 3A-3C illustrate a desirable recovery and conjugation of isolated antibodies using non-spiked 125 mM bicine and tricine, wherein Rabbit IgG conjugated to NHS DyLight® 488, mouse IgG conjugated to NHS DyLight® 680, and mouse IgG1 conjugated to NHS DyLight® 405 all exhibited desirable fluorescent signals.

Accordingly, this example demonstrates the amine buffer disclosed herein produces an isolated conjugatable antibody and subsequent conjugation to a fluorescent dye without further sample preparation steps.

### Example 4

In this example, bicine and tricine were investigating for producing conjugatable antibodies. Specifically, PanRas 10 (IgG2 A isotype) STAT3 (IgG2 A isotype), and HDAC 1 (rabbit polyclonal) were isolated using the buffers shown in Table 4 to equilibrate the matrix and subsequently conjugated to three different fluorescent dyes, NHS Alexa Fluor®647, NHS DyLight® 800, and NHS DyLight® 680 without any spiking.

FIG. 4A is a digital image of a fluorescent gel showing the recovery of isolated conjugatable PanRas 10, IgG2 A isotype subsequently conjugated to NHS Alexa Fluor®647 using non-spiked 125 mM bicine (Lane 1) and non-spiked 125 mM tricine (Lane 2); results demonstrate that bicine and tricine exhibited a desirable fluorescent signal for the PanRas 10 primary antibody-Alexa Fluor®647 conjugate. FIG. 4B is a digital image of a fluorescent gel showing the recovery of isolated conjugatable STAT3, IgG2 A isotype subsequently conjugated to NHS DyLight® 800 using non-spiked 125 mM bicine (Lane 1) and non-spiked 125 mM tricine (Lane 2); results demonstrate that bicine and tricine exhibited a desirable fluorescent signal for the STAT3 primary antibody-NHS DyLight® 800 conjugate. FIG. 4C is a digital image of a fluorescent gel showing the recovery of isolated conjugatable HDAC 1, rabbit polyclonal subsequently conjugated to NHS DyLight® 680 using non-spiked 125 mM bicine (Lane 1) and non-spiked 125 mM tricine (Lane 2); results demonstrate that bicine and tricine exhibited a desirable fluorescent signal for the HDAC primary antibody-NHS DyLight® 680 conjugate.

FIGS. 4A-4C demonstrate the recovery and conjugation of various antibodies using non-spiked 125 mM bicine and tricine. PanRas 10 IgG2 A conjugated to NHS Alexa Fluor®647, STAT3 IgG2 A conjugated to NHS DyLight® 800, and HDAC 1 rabbit polyclonal conjugated to NHS DyLight® 680 all exhibited desirable fluorescent signals. Accordingly, this example demonstrates that bicine and tricine in produced suitably-labeled, conjugatable antibodies with desirable fluorescent properties without further sample preparation steps.

### Example 5

In this example, bicine and tricine were investigating for producing conjugatable antibodies. Specifically, PanRas 10 (IgG2 A isotype), p 23 (IgG1 isotype), and STAT3 (IgG2 A isotope) were isolated using the buffers shown in Table 4 to equilibrate the matrix and subsequently conjugated to three different fluorescent dyes NHS DyLight® 650, NHS DyLight® 488, and NHS DyLight® 650 without any spiking.

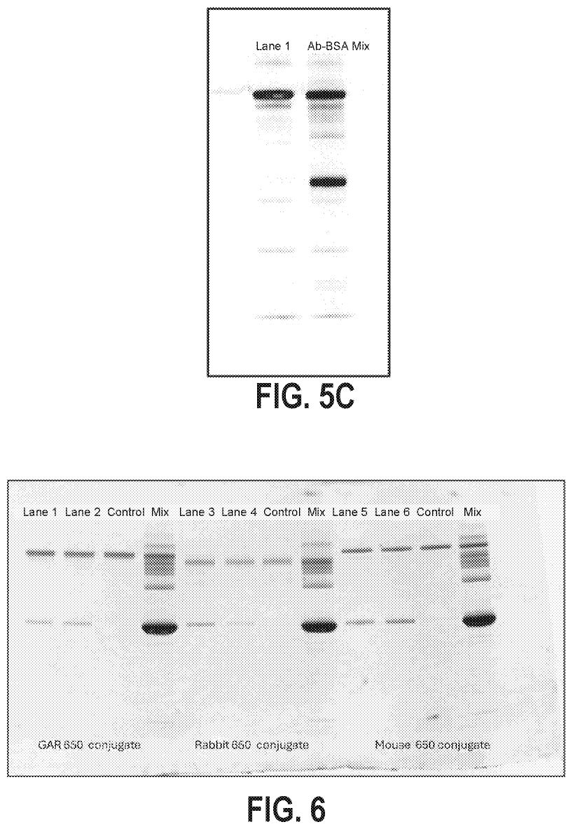

FIG. 5A is a digital image of a fluorescent gel showing the recovery of isolated conjugatable PanRas 10, IgG2 A isotype subsequently conjugated to NHS DyLight® 650 using non-spiked 125 mM bicine (Lane 1); results demonstrate that bicine and tricine exhibited a desirable fluorescent signal for the PanRas 10 primary antibody-NHS DyLight® 650 conjugate. FIG. 5B is a digital image of a fluorescent gel showing the recovery of isolated conjugatable p 23, IgG1 isotype subsequently conjugated to NHS DyLight® 488 using non-spiked 125 mM bicine (Lane 1); results demonstrate that bicine and tricine exhibited a desirable fluorescent signal for the p 23 primary antibody-NHS DyLight® 488 conjugate. FIG. 5C is a digital image of a fluorescent gel showing the recovery of isolated conjugatable STAT3, IgG2 A isotope subsequently conjugated to NHS DyLight® 650 using non-spiked 125 mM bicine (Lane 1); results demonstrate that bicine exhibited a desirable fluorescent signal for the STAT3 primary antibody-NHS DyLight® 650 conjugate.

In view of FIGS. 5A-5C, PanRas 10 IgG2 A conjugated to NHS DyLight® 650, p 23 IgG1 conjugated to NHS DyLight® 488, and STAT3 IgG2 A conjugated to NHS DyLight® 650 all exhibited strong fluorescent signals, demonstrating that bicine and tricine generated suitably labeled, conjugatable antibodies without further sample preparation steps.

### Example 6

In this example, the D/P ratio of cleaned-up conjugate using 125 mM bicine (pH 9.2) and subsequently conjugated with NHS DyLight® 650 and compared to D/P of the control (GAR, Rabbit, and mouse IgG) without BSA conjugated with NHS DyLight® 650 and the D/P of the antibodies-BSA mixture conjugated with NHS DyLight® 650.

Table 5 shows the D/P ratio of the cleaned-up conjugate using a matrix comprising (i) a porous size exclusion support and (ii) one or more cationic moieties associated with the porous size exclusion support equilibrated with 125 mM Bicine (pH 9.2) and subsequently conjugated with NHS DyLight® 650; and the D/P of the control (Mouse IgG) without BSA conjugated with NHS DyLight® 650 and the D/P of the Antibodies-BSA mix conjugated with NHS DyLight® 650.

TABLE 5

| | Lane | A 280 | A 655 | D/P |
|---|---|---|---|---|
| MS BSA Mix | Mix | 0.358 | 0.303 | 0.733 |
| (1 mg/mL-10 mg/mL) | | | | |
| MS (1 mg/mL) 650) - Control | Control | 0.059 | 0.158 | 2.51 |
| MS - cleaned (1) | 1 | 0.06 | 0.159 | 2.46 |
| MS - cleaned (2) | 2 | 0.065 | 0.175 | 2.8 |

Table 6 shows the D/P ratio of the cleaned-up conjugate a matrix comprising (i) a porous size exclusion support and (ii) one or more cationic moieties associated with the porous size exclusion support equilibrated with 125 mM Bicine (pH 9.2) and subsequently conjugated with NHS DyLight® 650; and the D/P of the control (GAR IgG) without BSA conjugated with NHS DyLight® 650 and the D/P of the Antibodies-BSA mix conjugated with NHS DyLight® 650.

TABLE 6

| | Lane | A 280 | A 655 | D/P |
|---|---|---|---|---|
| GAR BSA Mix | Mix | 0.321 | 0.26 | 0.7 |
| (1 mg/mL-10 mg/mL) | | | | |
| GAR (1 mg/mL) 650) - Control | Control | 0.046 | 0.141 | 3.31 |
| GAR - cleaned (1) | 3 | 0.041 | 0.126 | 2.92 |
| GAR - cleaned (2) | 4 | 0.042 | 0.218 | 2.88 |

Table 7 shows the D/P ratio of the cleaned-up conjugate using a matrix comprising (i) a porous size exclusion support and (ii) one or more cationic moieties associated with the porous size exclusion support equilibrated with 125 mM Bicine (pH 9.2) and subsequently conjugated with NHS DyLight® 650; and the D/P of the control (rabbit IgG) without BSA conjugated with NHS DyLight® 650 and the D/P of the Antibodies-BSA mix conjugated with NHS DyLight® 650.

TABLE 7

| | Lane | A 280 | A 655 | D/P |
|---|---|---|---|---|
| Rb BSA Mix | Mix | 0.308 | 0.269 | 0.758 |
| (1 mg/mL-10 mg/mL) | | | | |
| Rb (1 mg/mL) 650) - Control | Control | 0.052 | 0.139 | 2.5 |
| Rb - cleaned (1) | 5 | 0.051 | 0.133 | 2.42 |
| Rb - cleaned (2) | 6 | 0.046 | 0.122 | 2.48 |

Tables 5-7 show the D/P ratios of conjugates processed using a matrix comprising (i) a porous size exclusion support and (ii) one or more cationic moieties associated with the porous size exclusion support equilibrated with 125 mM bicine buffer (pH 9.2) were compared to controls. The cleaned-up conjugates and controls (GAR, Rabbit, and Mouse IgG) without BSA showed comparable D/P ratios. However, without being bound by one particular theory of operation, the Antibodies-BSA mix displayed low D/P ratios due to the dye reacting with BSA rather than the antibodies.

FIG. 6 is a digital image of a fluorescent gel showing the recovery of GAR 650 (left), rabbit 650 IgG (middle), and mouse 650 IgG (right) conjugated to NHS DyLight® 650 using non-spiked 125 mM bicine (Lane 1); results demonstrate a desirable recovery of GAR 650, rabbit 650 IgG, and mouse 650 IgG conjugated with NHS DyLight® 650 using non-spiked 125 mM bicine.

Accordingly, this example demonstrates desirable recovery for all three antibodies and efficient conjugation using non-spiked 125 mM bicine without further sample preparation steps.

### Example 7

In this example, non-spiked 25 mM bicine (pH 9.2), 25 mM bicine (pH 9.2) spiked with 1 M borate (9.4), non-spiked 75 mM sodium acetate (pH 7.0), and 75 mM sodium acetate (pH 7.0) spiked with 1 M borate (pH 9.4) were used for equilibrating a matrix comprising a porous size exclusion support and cationic moiety and were investigated for removing BSA and producing isolated conjugatable antibody from sample.

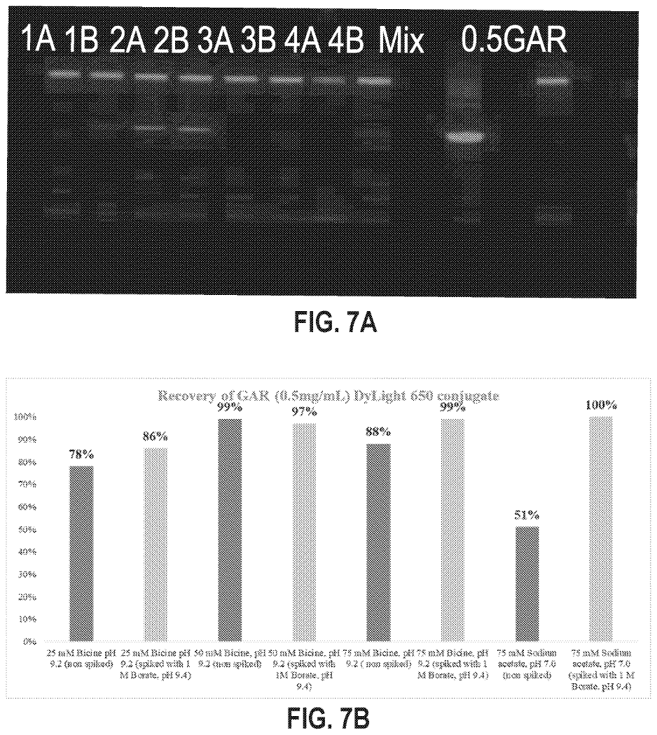

FIG. 7A is a digital image of a fluorescent gel showing the recovery of isolated conjugatable GAR (0.5 mg/mL) subsequently conjugated to NHS DyLight® 650 using non-spiked 25 mM bicine (pH 9.2) (Lane 1A); 25 mM bicine (pH 9.2) spiked with 1 M borate (pH 9.4) (Lane 1B); non-spiked 50 mM bicine (pH 9.2) (Lane 2A); 50 mM bicine (pH 9.2) spiked with 1 M borate (pH 9.4) (Lane 2B); non-spiked 75 mM bicine (pH 9.2) (Lane 3A); 75 mM bicine (pH 9.2) spiked with 1 M borate (pH 9.4) (Lane 3B); non-spiked 75 mM sodium acetate (pH 7.0) (Lane 4A); and 75 mM sodium acetate (pH 7.0) spiked with 1 M borate (pH 9.4) (Lane 4B); demonstrating that non-spiked bicine exhibited a desirable fluorescent signal for the GAR antibody-NHS DyLight® 650 conjugate unlike the non-spiked sodium acetate.

FIG. 7B is a bar graph showing the GAR-Dy 650 recovery (%) of the fluorescent gel of FIG. 7A and demonstrating a 78% recovery for non-spiked 25 mM bicine (pH 9.2) (Lane 1A); 86% recovery for 25 mM bicine spiked with 1 M borate (pH 9.4) (Lane 1 B); 99% recovery for non-spiked 50 mM bicine (pH 9.2) (Lane 2A); 97% recovery for 50 mM bicine spiked with 1 M borate (pH 9.4) (Lane 2B); 88% recovery for non-spiked 75 mM bicine (pH 9.2) (Lane 3A); 99% recovery for 75 mM bicine spiked with 1 M borate (pH 9.4) (Lane 3B); 51% recovery for non-spiked 75 mM sodium acetate (pH 7.0) (Lane 4A); and 100% recovery for 75 mM sodium acetate spiked with 1 M borate (pH 9.4) (Lane 4B).

FIGS. 7A-7B show that bicine can be used for resin equilibration and isolation of conjugatable antibodies. In contrast, sodium acetate required an additional step of borate spiking to achieve suitable recovery percentages for the GAR-DyLight® 650 conjugate.

Accordingly, this example demonstrates that the amine buffer disclosed herein facilitates the isolation of conjugatable antibodies and their conjugation to fluorescent dyes, eliminating the need for additional sample preparation steps.

### Example 8

In this example, 125 mM bicine (pH 9.2) was used to equilibrate a spin column comprising a matrix comprising (i) a porous size exclusion support and (ii) one or more cationic moieties associated with the porous size exclusion support equilibrated with (0.5 mL spin column with 50 μL sample load) and investigated for producing conjugatable antibodies and a desirable fluorescent conjugate signal. After BSA removal, the isolated conjugatable antibody Rabbit IgG was conjugated with DyLight® 650 without any prior buffer exchange step using borate buffer.

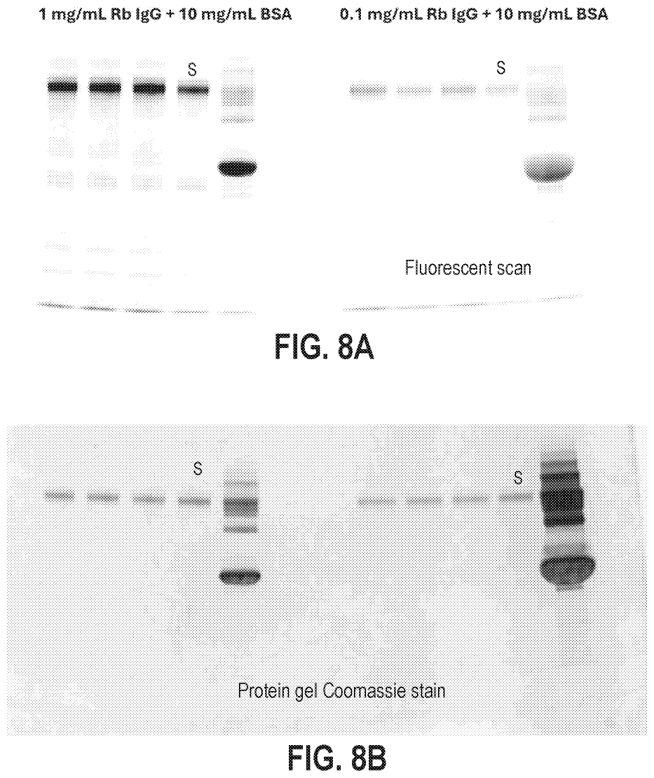

FIG. 8A is a digital image of a fluorescent gel (fluorescent scan) showing the recovery of isolated conjugatable rabbit IgG subsequently conjugated to NHS DyLight® 650 (Lane start “S” is the borate buffer exchanged using a 40 K resin subsequently conjugated with NHS Dy 650) from a sample comprising 1 mg/mL rabbit IgG and 10 mg/mL BSA (left) and from a sample comprising 0.1 mg/mL rabbit IgG and 10 mg/mL BSA (right); results demonstrate that bicine exhibited a desirable fluorescent signal for the rabbit IgG antibody-NHS DyLight® 650 conjugate.

FIG. 8B is a digital image of a Coomassie protein-stained gel showing the recovery of isolated conjugatable rabbit IgG subsequently conjugated to NHS DyLight® 650 (Lane start “S” is the borate buffer exchanged using a 40 K resin subsequently conjugated with NHS Dy 650) from a sample comprising 1 mg/mL rabbit IgG and 10 mg/mL BSA (left) and from a sample comprising 0.1 mg/mL rabbit IgG and 10 mg/mL BSA (right); results demonstrate that bicine exhibited desirable rabbit IgG antibody-NHS DyLight® 650 conjugate recovery.

This example demonstrates the use of 125 mM bicine (pH 9.2) for equilibrating a spin column with a matrix comprising (i) a porous size exclusion support and (ii) one or more cationic moieties associated with the porous size exclusion support, enabling the production of conjugatable Rabbit IgG antibodies without the need for prior buffer exchange using borate. Moreover, 125 mM bicine (pH 9.2) a desirable fluorescent signal and recovery of the conjugate.

### Example 9

In this example, 125 mM bicine (pH 9.2) was used to equilibrate a matrix comprising (i) a porous size exclusion support and (ii) one or more cationic moieties associated with the porous size exclusion support and investigated for producing conjugatable antibodies and a desirable fluorescent conjugate signal. After BSA removal, the isolated conjugatable antibodies RAS 10 and STAT3 were conjugated with DyLight® 650 without any prior buffer exchange step using borate buffer; and isolated conjugatable p23 was conjugated with NHS DyLight® 488 without any prior buffer exchange steps using borate buffer.

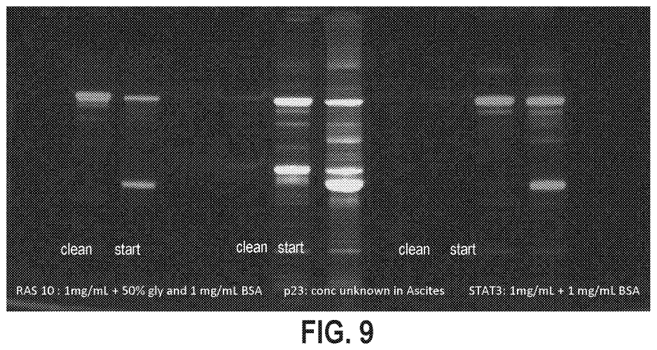

FIG. 9 is a digital image of a fluorescent gel showing the recovery of isolated conjugatable RAS 10 subsequently conjugated to NHS DyLight® 650 from a sample comprising 1 mg/mL Ras 10, 50% gly, 1 mg/mL BSA (left); isolated conjugatable p 23 subsequently conjugated to NHS DyLight® 488 (middle); isolated conjugatable STAT3 subsequently conjugated to NHS DyLight® 650 from a sample comprising 1 mg/mL STAT3 and 1 mg/mL BSA (right); results demonstrate that bicine produced isolated conjugatable primary antibodies RAS 10, p23, and STAT3 and subsequent conjugation produced a desirable fluorescent signal.

This example demonstrates that after BSA removal, the antibodies RAS 10 and STAT3 were conjugated with DyLight® 650, and p23 was conjugated with NHS DyLight® 488, all generating desirable conjugatable antibodies and fluorescent conjugates, without any buffer exchange using borate.

### Example 10

In this example, a spin column comprising a matrix comprising (i) a porous size exclusion support and (ii) one or more cationic moieties covalently bound with the porous size exclusion support was equilibrated with 5 mM bicine, 80 mM NaCl, pH 9.0 and used to isolate conjugatable human IgG from human serum. The spin column had a 10 mL resin bed, and the human serum sample had a volume of 400 μL and was diluted 1:1. The isolated conjugatable human IgG was subsequently conjugated to NHS DyLight® 650 without prior buffer exchange or dialysis.

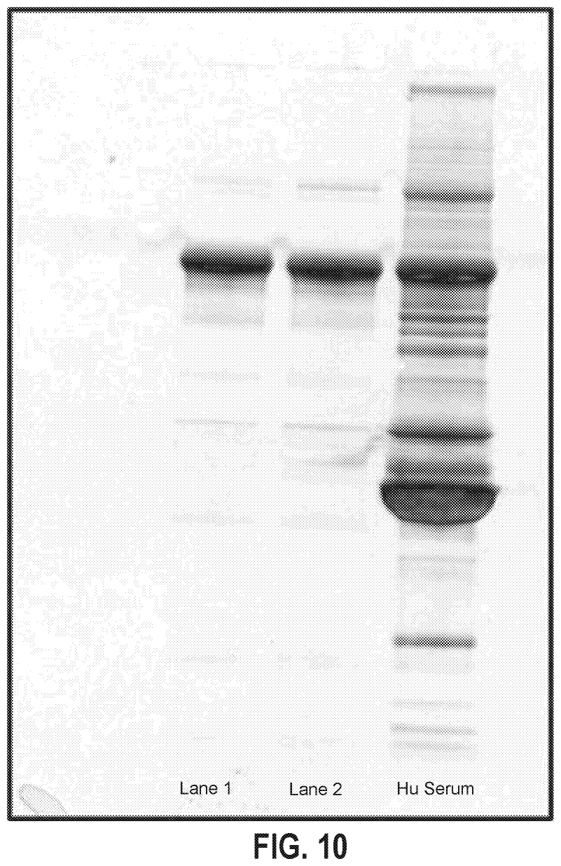

FIG. 10 is a digital image of a fluorescent gel showing the recovery of isolated conjugatable human IgG from human serum using the matrix, wherein Lanes 1 and 2 are duplicate isolated samples from human serum and the human serum starting material is shown in Lane 3. The results demonstrate that the matrix produced isolated conjugatable human IgG.

This isolated conjugatable human IgG was then successfully conjugated to NHS DyLight® 650 and produced a desirable fluorescent signal without requiring buffer exchange or dialysis prior to conjugation.

### Example 11

In this example, a spin column comprising a matrix comprising (i) a porous size exclusion support and (ii) one or more cationic moieties covalently bound with the porous size exclusion support was equilibrated with 125 mM bicine, 100 mM NaCl, pH 9.0 and used to isolate conjugatable antibodies from antibody-containing samples. The spin column had a 2 mL format and a 100 μL sample load. BSA was removed from the antibody-containing samples, and the isolated conjugatable antibodies Goat Anti-Rabbit, Rat IgG, and Mouse IgG were subsequently conjugated to DyLight® 488, DyLight® 800, and DyLight® 650, respectively, without prior buffer exchange or dialysis.

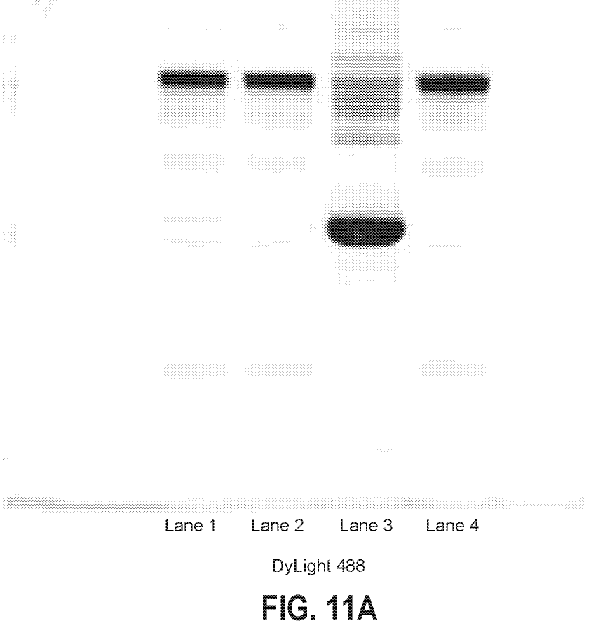

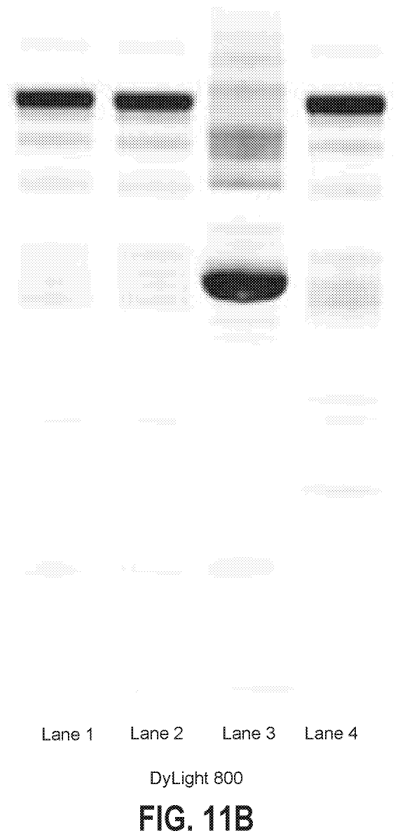

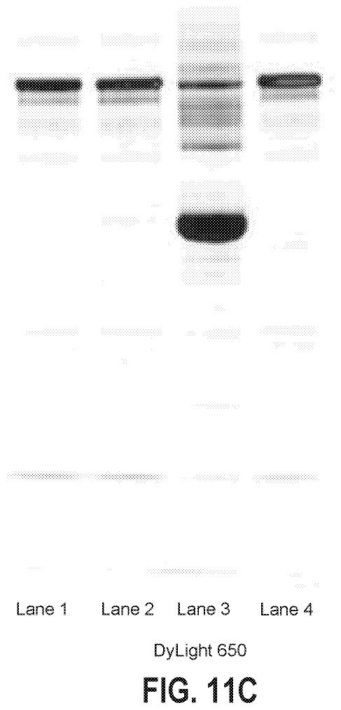

FIG. 11A is a digital image of a gel showing results from using the matrix in combination with bicine and the Goat Anti-Rabbit antibody, wherein Lanes 1 and 2 show duplicate samples purified from an antibody-BSA mixture using the matrix, Lane 3 shows the antibody-BSA mixture prior to cleanup, and Lane 4 shows a control antibody sample without BSA. The results shown in FIG. 11A demonstrate that using bicine with the matrix facilitates isolating a conjugatable form of the Goat Anti-Rabbit antibody, which after conjugation with the DyLight® 488, produces a detectable fluorescent signal, all without having to perform additional sample preparation and/or cleaning steps prior to conjugation.

FIG. 11B is a digital image of a gel showing results from using the matrix in combination with bicine and the Rat IgG antibody, wherein Lanes 1 and 2 show duplicate samples purified from an antibody-BSA mixture using the matrix, Lane 3 shows the antibody-BSA mixture prior to cleanup, and Lane 4 shows a control antibody sample without BSA. The results shown in FIG. 11B demonstrate that using bicine with the matrix facilitates isolating a conjugatable form of the Rat IgG antibody, which after conjugation with the DyLight® 800, produces a detectable fluorescent signal, all without having to perform additional sample preparation and/or cleaning steps prior to conjugation.

FIG. 11C is a digital image of a gel showing results from using the matrix in combination with bicine and the Mouse IgG antibody, wherein Lanes 1 and 2 show duplicate samples purified from an antibody-BSA mixture using the matrix, Lane 3 shows the antibody-BSA mixture prior to cleanup, and Lane 4 shows a control antibody sample without BSA. The results shown in FIG. 11C demonstrate that using bicine with the matrix facilitates isolating a conjugatable form of the Mouse IgG antibody, which after conjugation with the DyLight® 650, produces a detectable fluorescent signal, all without having to perform additional sample preparation and/or cleaning steps prior to conjugation.

In summary, this example demonstrates that bicine can be used with the matrix to successfully isolate conjugatable antibodies from serum-containing samples by removing BSA from the antibody-containing samples, thereby enabling subsequent conjugation of the antibodies to fluorescent labels without further sample preparation or cleaning steps. Such results would similarly be expected for other types of antibodies (e.g., human antibodies) and samples (e.g., human serum).

In view of the many possible aspects to which the principles of the present disclosure may be applied, it should be recognized that the illustrated aspects are only preferred examples of the present disclosure and should not be taken as limiting the scope of the present disclosure. Rather, the scope of the present disclosure is defined by the following claims. We therefore claim as our invention all that comes within the scope and spirit of these claims.

We claim:

1. A method, comprising:
combining an amine buffer according to Formula I with a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof,
wherein Formula I is 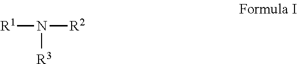
wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof.

2. The method of claim 1, wherein the amine buffer is a secondary amine or a tertiary amine.

3. The method of claim 2, wherein the amine buffer is a secondary amine and at least one of R¹, R², and R³ is a heteroaliphatic group.

4. The method of claim 3, wherein the amine buffer is tricine.

5. The method of claim 2, wherein the amine buffer is a tertiary amine and two of R¹, R², and R³ independently are a heteroaliphatic group.

6. The method of claim 5, wherein the amine buffer is bicine.

7. The method of claim 1, wherein the amine buffer has a pH ranging from 7.0 to 9.5.

8. The method of claim 1, further comprising adding a dye.

9. The method of claim 8, wherein the dye is added after the one or more molecules are isolated.

10. The method of claim 8, wherein the dye is a fluorescent dye.

11. A method, comprising:
exposing a sample to a matrix and a buffer, wherein
(a) the matrix comprises (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof; and
(b) the buffer is an amine buffer according to Formula I, 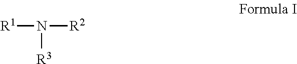
wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof.

12. The method of claim 11, wherein the sample is exposed to a composition comprising the matrix and the buffer.

13. The method of claim 11, wherein (i) the sample is first exposed to the matrix and then exposed to the buffer, or (ii) the sample is first exposed to the buffer and then exposed to the matrix.

14. The method of claim 11, wherein the sample comprises an antibody.

15. The method of claim 14, further comprising isolating the antibody.

16. The method of claim 15, further comprising adding a dye to facilitate forming a conjugate with the antibody.

17. The method of claim 16, wherein the dye is a fluorescent dye.

18. The method of claim 11, wherein the buffer is bicine.

19. The method of claim 11, wherein the buffer is tricine.

20. A composition, comprising:
an amine buffer according to Formula I, 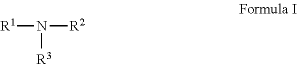
wherein each of R¹, R², and R³ independently is selected from hydrogen, an aliphatic group, a heteroaliphatic group, or comprises a carboxylic acid or an anion thereof, provided that one of R¹, R², and R³ comprises the carboxylic acid or the anion thereof; and
a matrix comprising (i) a porous size exclusion support, and (ii) one or more cationic moieties associated with the porous size exclusion support, wherein the matrix separates one or more molecules in a sample based on a molecular weight of the one or more molecules, a charge of the one or more molecules, an isoelectric point (pI) of the one or more molecules, or a combination thereof.

21. The composition of claim 20, further comprising an antibody, a dye, or both.

22. The composition of claim 20, wherein the porous size exclusion support has a molecular weight cut-off of greater than or equal to 40 kDa.

23. The composition of claim 20, wherein the amine buffer is a secondary amine or a tertiary amine.

24. The composition of claim 23, wherein the amine buffer is a secondary amine and at least one of R¹, R², and R³ is a heteroaliphatic group; or the amine buffer is a tertiary amine and two of R¹, R², and R³ independently are a heteroaliphatic group.

25. The composition of claim 24, wherein the amine buffer is bicine or tricine.

26. A kit comprising the composition of claim 20, wherein the kit comprises a first container comprising the amine buffer and a second container comprising the matrix.
